(19) United States
(12) Patent Application Publication
(10) Pub. No.: US 2026/0283241 A1
(43) Pub. Date: Sep. 24, 2026

(54) PERSONAL VAPORIZERS WITH ISOLATED WICK STRUCTURES

(71) Applicant: BLACKSHIP TECHNOLOGIES DEVELOPMENT LLC, Richmond, VA (US)

(72) Inventors: Yongjie XU, Richmond, VA (US); Donovan PHILLIPS, Richmond, VA (US)

(21) Appl. No.: 19/476,489

(22) PCT Filed: Apr. 18, 2024
(86) PCT No.: PCT/US2024/025159

§ 371 Date: Oct. 17, 2025

Related U.S. Application Data
(60) Provisional application No. 63/497,288, filed on Apr. 20, 2023.

(51) Int. Cl. A24F 40/44; A24F 40/10; A24F 40/42; A24F 40/46; A24F 40/485; A61M 15/06

(52) U.S. Cl. CPC A24F 40/44; A24F 40/10; A24F 40/42; A24F 40/46; A24F 40/485; A61M 15/06

(57) ABSTRACT

A personal vaporizer has a main body in which are disposed a vaporization chamber defined by a vaporization chamber wall, a selectively operable heating element disposed within the vaporization chamber, a fluid reservoir surrounding at least a portion of the vaporization chamber, a fluid flow window through the vaporization chamber wall between the reservoir and the vaporization chamber, a wick body having an upstream surface and a downstream surface, and a vaporizable liquid disposed within the fluid reservoir and isolated from the wick body. A method of operating the personal vaporizer includes establishing fluid communication between the fluid reservoir and the upstream surface of the wick body via the fluid flow window, allowing vaporizable liquid to flow to and through the wick body, and powering the heating element to vaporize vaporizable liquid in the vaporization chamber.

### BACKGROUND OF THE INVENTION

The invention relates generally to micro-vaporizer wicking materials and, more particularly, to composite micro-vaporizer wicks formed from a fiber material and an active flavor material. Another aspect of the invention relates to personal vaporizers, and more particularly to personal vaporizers in which a fluid transport structure is isolated from all vaporizer liquid until just before use.

Micro-vaporizers are devices in which a vaporizable fluid is drawn from a storage reservoir into a chamber where it is heated to vaporization temperature by a heating element. The vaporized fluid is then drawn or forced from the chamber. In products such as electronic cigarettes (also known as e-cigarettes or personal vaporizers), the vaporized fluid is drawn from the chamber through a mouthpiece and inhaled by the user. In other products the vaporized fluid is dispersed into the atmosphere.

The usual purpose of a device that uses a micro-vaporizer is to dispense one or more active substances using the vaporized fluid. In atmospheric dispensers, these substances may include materials such as deodorizing agents, fragrance, and insect repellant. In the case of personal vaporizers, the active substances typically include a flavorant (i.e., a flavoring agent or material) and nicotine. The flavorant and nicotine levels may be selected so as to mimic the experience of smoking a cigarette. In general, the vaporizable fluid is the sole source of active substances exiting the micro-vaporizer.

### SUMMARY OF SOME EMBODIMENTS OF THE INVENTION

An illustrative aspect of the invention provides a composite wick for use in a micro-vaporizer. The micro-vaporizer has a micro-vaporizer body in which is disposed a vaporizable fluid reservoir and a vaporization chamber with a heating element at least partially disposed therein. The composite wick comprises a wick body having an upstream surface and a downstream surface. The wick body is positionable within the micro-vaporizer body so that the upstream surface is in fluid communication with the vaporizable fluid reservoir and the downstream surface is disposed within the vaporization chamber in opposition to a surface of the heating element. The wick body comprises at least one base wick structure having a plurality of tortuous passages that collectively provide a capillary effect to draw vaporizable fluid from the vaporizable fluid reservoir and transport it toward the downstream surface. The wick body further comprises an active material positioned so that vaporizable fluid drawn through the wick body contacts and interacts with the active material. The active material is selected to impart a desired characteristic to the vaporizable fluid.

Another illustrative aspect of the invention provides a method of modifying vaporization products produced in a micro-vaporizer. The micro-vaporizer has a micro-vaporizer body in which is disposed a vaporizable fluid reservoir and a vaporization chamber with a selectively activated heating element at least partially disposed therein. The method comprises positioning a composite wick intermediate the vaporizable fluid reservoir and the heating element for transporting vaporizable fluid therebetween. The composite wick comprises a base wick structure configured to draw vaporizable fluid toward the downstream surface of the wick and an active material selected to impart a desired characteristic to the vaporizable fluid. The method further comprises allowing vaporizable fluid to flow from the reservoir into and through the composite wick, thereby causing the vaporizable fluid to interact with the active material to produce a modified vaporizable fluid mixture. The method also comprises activating the heating element to vaporize the modified vaporizable fluid mixture at or near the downstream surface of the wick, thereby producing modified vaporization products.

Another illustrative aspect of the invention provides vaporizers that incorporate composite wicks according to embodiments of the invention. Many of these vaporizers incorporate mechanisms for isolating composite wick elements from vaporizable fluid prior to first use of said vaporizers and for selectively establishing fluid communication between the composite wick elements and the vaporizable fluid when placed in operating configurations.

Another illustrative aspect of the invention provides a personal vaporizer comprising a main body having a main body wall defining a main body interior. The personal vaporizer further comprises a vaporization chamber disposed within the main body interior. The vaporization chamber is defined at least in part by a vaporization chamber wall having a fluid flow window formed therethrough. The personal vaporizer further comprises an exit chamber extending from the vaporization chamber to an exit passage in communication with an atmosphere exterior to the main body. The personal vaporizer still further comprises a heating element disposed within the vaporization chamber, a fluid reservoir, and a fluid transport structure. The fluid reservoir has a reservoir interior defined at least in part by the vaporization chamber wall and is configured for storage of a vaporizable liquid therein. The fluid reservoir is also configured for passage of the vaporizable liquid through the fluid flow window formed through the vaporization chamber wall when the personal vaporizer is in an operational configuration. The fluid transport structure comprises a wick body at least partially disposed within the vaporization chamber. The wick body is configured for drawing vaporizable liquid through an upstream wick surface adjacent or disposed within the fluid flow window when the personal vaporizer is in the operational configuration and for transporting the vaporizable liquid to a downstream wick surface in contact with or adjacent the heating element. The personal vaporizer also comprises one or more impermeable fluid barriers configured and positioned to prevent vaporizable liquid from passing through the fluid flow window when the personal vaporizer is in a pre-use configuration and for allowing vaporizable liquid to pass through the fluid flow window and contact the upstream wick surface when the personal vaporizer is in the operational configuration.

Another illustrative aspect of the invention provides a personal vaporizer comprising a wick cartridge that comprises a cylindrical wick casing having at least one wick casing window formed therethrough, an annular wick body disposed within the cylindrical wick casing, and a heating element. The annular wick body has an inner cylindrical surface and an outer cylindrical surface and is configured for receiving vaporizable fluid through the outer cylindrical surface and conducting it through the cylindrical wick body to the inner cylindrical surface. The heating element is at least partially disposed within the annular wick body so that at least a portion of the heating element is in contact with or adjacent the inner cylindrical surface of the annular wick body. The personal vaporizer further comprises a main vaporizer body having a main body wall defining a main body interior and having a longitudinal axis, a vaporization chamber, and a fluid reservoir. The vaporization chamber is disposed within the main body interior and is defined by a cylindrical vaporization chamber wall surrounding the longitudinal axis. The vaporization chamber wall has at least one fluid flow window formed therethrough. The vaporization chamber is configured for receiving the wick cartridge so that the at least one fluid window is in registration with the at least one wick casing window when the wick cartridge is fully received into an installed position within the vaporization chamber. The fluid reservoir surrounds at least a portion of the vaporization chamber and is configured for storage of a vaporizable liquid therein. The fluid reservoir is in fluid communication with the at least one fluid flow window. The personal vaporizer also comprises a plug member for each of the at least one fluid flow window. Each plug member is positioned at least partially within its respective fluid flow window and extending into the vaporization chamber. Each plug member is configured to prevent fluid flow through its respective fluid flow window until the wick cartridge has been received into the vaporization chamber.

Another aspect of the invention provides a method of operating a personal vaporizer having a main body defining a main body interior, a vaporization chamber defined within the main body interior by a vaporization chamber wall, a selectively operable heating element disposed within the vaporization chamber, a fluid reservoir surrounding at least a portion of the vaporization chamber, a fluid flow window through the vaporization chamber wall between the reservoir and the vaporization chamber, a wick body having an upstream surface and a downstream surface, and a vaporizable liquid disposed within the fluid reservoir and isolated from the wick body. The method comprises establishing fluid communication between the fluid reservoir and the upstream surface of the wick body via the fluid flow window, allowing vaporizable liquid to flow to and through the wick body, and powering the heating element to vaporize vaporizable liquid in the vaporization chamber.

Another aspect of the invention includes a personal vaporizer comprising: a main body having a main body wall defining a main body interior; a vaporization chamber within the main body interior, the vaporization chamber being defined at least in part by a vaporization chamber wall having a fluid flow window formed therethrough; an exit chamber extending from the vaporization chamber to an exit passage in communication with an atmosphere exterior to the main body; a heating element disposed within the vaporization chamber; a fluid reservoir having a reservoir interior defined at least in part by the vaporization chamber wall, the fluid reservoir being configured for storage of a vaporizable liquid therein and for passage of the vaporizable liquid through the fluid flow window formed through the vaporization chamber wall when the personal vaporizer is in an operational configuration; a fluid transport structure comprising a wick body at least partially disposed within the vaporization chamber and configured for drawing vaporizable liquid through an upstream wick surface adjacent or disposed within the fluid flow window when the personal vaporizer is in the operational configuration and for transporting the vaporizable liquid to a downstream wick surface in contact with or adjacent the heating element; and one or more impermeable fluid barriers configured and positioned to prevent vaporizable liquid from passing through the fluid flow window when the personal vaporizer is in a pre-use configuration and for allowing vaporizable liquid to pass through the fluid flow window and contact the upstream wick surface when the personal vaporizer is in the operational configuration.

The personal vaporizer can be selectively changed from the pre-use configuration to the operational configuration by at least one of the set consisting of removal, displacement, distortion, or breach of at least one of the one or more impermeable fluid barriers. The main body is cylindrical, has a main body longitudinal axis, and is positionable in a vertical orientation in which the main body longitudinal axis is vertically aligned, the vaporization chamber is cylindrical and has a longitudinal chamber axis that is coaxial with the main body longitudinal axis, the wick body is an annular cylinder surrounding the heating element with the downstream surface being an inner cylindrical surface of the annular cylinder that is in contact with or adjacent the heating element and the upstream surface is an outer cylindrical surface of the annular cylinder, the fluid reservoir has an upper reservoir portion and a lower reservoir portion, the lower reservoir portion being in fluid communication with the fluid flow window through the cylindrical vaporization chamber wall, and the one or more impermeable fluid barriers comprises a fluid impermeable barrier member positioned between the upper and lower reservoir portions and configured to prevent vaporizable liquid in the upper reservoir portion from flowing into the lower reservoir portion until the fluid impermeable barrier member is removed or breached.

A tab portion of the fluid impermeable barrier member extends through an opening in the case wall and the fluid impermeable barrier member is configured so that it can be removed from the main body by grasping the tab portion and pulling the barrier member through the opening. The fluid impermeable barrier member is a flexible membrane. The personal vaporizer further comprises penetration means configured for selectively breaching the fluid impermeable barrier member. The main body has an upper body portion in which the upper reservoir portion is disposed and a lower body portion in which the lower reservoir portion is disposed, the lower body portion being axially movable relative to the upper body portion from a pre-use position to a use position, the fluid impermeable barrier member is fixedly attached to the upper body portion, and the personal vaporizer further comprises: at least one barrier contact member attached to the lower body portion, the at least one barrier contact member being configured and positioned so that it is spaced apart from the fluid impermeable barrier member when the lower body portion is in the pre-use position and so that when the lower body portion is moved to the use position, the at least one barrier contact member contacts and breaches the fluid impermeable barrier member, thereby permitting vaporizable liquid to flow from the upper reservoir portion into the lower reservoir portion.

The lower body portion is threadably attached to the upper body portion so that axial motion of the lower body portion relative to the upper body portion is accomplished by rotation of the lower body portion relative to the upper body portion. The lower body portion is at least partially telescopically receivable into the upper body portion. The main body is cylindrical, has a main body longitudinal axis, and has an upper body portion having an upper body wall defining an upper body interior and a lower body portion having a lower body wall defining a lower body interior, the lower body portion being movably attached to the upper body portion to allow movement of the lower body portion relative to the upper body portion from a pre-use position to a use position, the vaporization chamber is cylindrical and has a longitudinal chamber axis that is coaxial with the main body longitudinal axis, the wick body is an annular cylinder surrounding the heating element with the downstream surface being an inner cylindrical surface of the annular cylinder that is in contact with or adjacent the heating element and the upstream surface is an outer cylindrical surface of the annular cylinder, the fluid transport structure further comprises a cylindrical wick casing surrounding the wick body and having at least one wick casing window formed therethrough, the wick casing being attached to the lower body portion and configured so that when the lower case portion is in the pre-use position, the at least one wick casing window is out of registration with the at least one fluid flow window and so that when the lower body portion is moved to the use position, the at least one wick casing window moves into registration with the at least one fluid flow window.

The personal vaporizer further comprises: a locking mechanism configured to engage the lower body portion when the lower body portion is moved into the use position and to prevent movement of the lower body portion away from the use position. The lower body portion is movable from the pre-use position to the use position by rotating the upper body portion relative to the lower body portion. The lower body portion is movable from the pre-use position to the use position by translating the upper body portion relative to the lower body portion along the longitudinal axis.

Another aspect of the invention includes a personal vaporizer comprising: a wick cartridge comprising: a cylindrical wick casing having at least one wick casing window formed therethrough; an annular wick body disposed within the cylindrical wick casing, the annular wick body having an inner cylindrical surface and an outer cylindrical surface and being configured for receiving vaporizable fluid through the outer cylindrical surface and conducting it through the cylindrical wick body to the inner cylindrical surface; and a heating element at least partially disposed within the annular wick body so that at least a portion of the heating element is in contact with or adjacent the inner cylindrical surface of the annular wick body; a main vaporizer body having a main body wall defining a main body interior and having a longitudinal axis; a vaporization chamber within the main body interior, the vaporization chamber being defined by a cylindrical vaporization chamber wall surrounding the longitudinal axis and having at least one fluid flow window formed therethrough, the vaporization chamber being configured for receiving the wick cartridge so that the at least one fluid window is in registration with the at least one wick casing window when the wick cartridge is fully received into an installed position within the vaporization chamber; a fluid reservoir surrounding at least a portion of the vaporization chamber and configured for storage of a vaporizable liquid therein, the fluid reservoir being in fluid communication with the at least one fluid flow window; and a plug member for each of the at least one fluid flow window, each plug member being positioned at least partially within its respective fluid flow window and extending into the vaporization chamber and being configured to prevent fluid flow through its respective fluid flow window until the wick cartridge has been received into the vaporization chamber.

The personal vaporizer further comprises: a locking mechanism configured to engage and retain the wick cartridge when the wick cartridge is in the installed position. Each plug member is configured and positioned so that when the wick cartridge is received into the vaporization chamber, the wick casing contacts the plug member and at least partially displaces the plug member such that fluid is allowed to pass through the at least one fluid flow window. Each plug member is configured and positioned so that when the wick cartridge is received into the vaporization chamber, the wick casing contacts the plug member and at least partially distorts the plug member such that fluid is allowed to pass through the at least one fluid flow window. Each plug member comprises a frangible portion configured and positioned so that when the wick cartridge is received into the vaporization chamber, the wick casing contacts and fractures the frangible portion so that fluid is allowed to pass through the at least one fluid flow window.

Another aspect of the invention includes a method of operating a personal vaporizer having a main body defining a main body interior, a vaporization chamber defined within the main body interior by a vaporization chamber wall, a selectively operable heating element disposed within the vaporization chamber, a fluid reservoir surrounding at least a portion of the vaporization chamber, a fluid flow window through the vaporization chamber wall between the reservoir and the vaporization chamber, a wick body having an upstream surface and a downstream surface, and a vaporizable liquid disposed within the fluid reservoir and isolated from the wick body, the method comprising: establishing fluid communication between the fluid reservoir and the upstream surface of the wick body via the fluid flow window; allowing vaporizable liquid to flow to and through the wick body; and powering the heating element to vaporize vaporizable liquid in the vaporization chamber.

The fluid reservoir has upper and lower reservoir portions separated by a liquid impermeable barrier, the vaporizable liquid is initially disposed only within the upper reservoir portion, and the action of establishing fluid communication includes: removing the liquid impermeable barrier, thereby establishing fluid communication between the upper and lower reservoir portions, and placing the personal vaporizer in a vertical position with the upper reservoir portion above the lower reservoir portion, thereby causing the vaporizable liquid to flow into the lower reservoir portion and through the fluid flow window to the upstream surface of the wick body.

The fluid reservoir has upper and lower reservoir portions separated by a liquid impermeable barrier, the vaporizable liquid is initially disposed only within the upper reservoir portion, and the action of establishing fluid communication includes: breaching the liquid impermeable barrier, thereby establishing fluid communication between the upper and lower reservoir portions, and placing the personal vaporizer in a vertical position with the upper reservoir portion above the lower reservoir portion, thereby causing the vaporizable liquid to flow into the lower reservoir portion and through the fluid flow window to the upstream surface of the wick body. The main body has an upper body portion in which the upper reservoir portion is disposed and a lower body portion in which the lower reservoir portion is disposed, the lower body portion being axially movable relative to the upper body portion from a pre-use position to a use position, the fluid impermeable barrier member is fixedly attached to the upper body portion, a barrier contact member is attached to the lower body portion for movement therewith, the barrier contact member being configured to contact and breach the fluid impermeable barrier member when the lower body portion is moved from the pre-use position to the use position, and the action of breaching the liquid impermeable barrier includes moving the lower body portion from the pre-use position to the use position, thereby causing the contact member to contact and breach the liquid impermeable barrier.

The method further comprises: locking the lower body portion in the use position to prevent further movement of the lower body portion relative to the upper body portion. The lower body portion is threadably attached to the upper body portion, and the action of moving the lower body portion includes rotating the lower body portion relative to the upper body to produce axial movement of the lower body portion relative to the upper body. The main body has an upper body portion and a lower body portion movably attached to the upper body portion to allow movement of the lower body portion relative to the upper body portion from a pre-use position to a use position, the fluid reservoir and the fluid flow window are disposed within the upper body interior, the wick body is disposed within a wick casing that is attached to the lower body portion and has a wick casing window, the wick casing being configured so that when the lower case portion is in a pre-use position, the wick casing window is out of registration with the fluid flow window and when the lower body portion is moved to a use position, the wick casing window moves into registration with the fluid flow window so that the fluid reservoir is in fluid communication with the upstream surface of the wick body via the fluid flow window and the wick casing window, and the action of establishing fluid communication includes: moving the lower body portion from the pre-use position to the use position.

The method further comprises: locking the lower body portion in the use position to prevent further movement of the lower body portion relative to the upper body portion. The action of moving the lower body portion from the pre-use position to the use position includes rotating the upper body portion relative to the lower body portion around a longitudinal axis common to the upper and lower body portions. The action of moving the lower body portion from the pre-use position to the use position includes translating the upper body portion relative to the lower body portion along a longitudinal axis common to the upper and lower body portions The personal vaporizer comprises a wick cartridge that includes the wick body, a wick casing having a casing window, and the heating element, the wick body being disposed within the cylindrical wick casing, the vaporization chamber is configured for receiving the wick cartridge so that the fluid window is in registration with the wick casing window when the wick cartridge is fully received into an installed position within the vaporization chamber, the personal vaporizer comprises a plug member positioned at least partially within the fluid flow window, the plug member having an initial configuration in which a portion of the plug member extends into the vaporization chamber and the plug member is positioned to prevent fluid flow through the fluid flow window until the wick cartridge is received into the vaporization chamber, and the action of establishing fluid communication includes: inserting the wick cartridge into the vaporization chamber, and placing the wick cartridge in the installed position. The plug member is configured and positioned so that when the wick cartridge is received into the vaporization chamber, the wick casing contacts the plug member and at least partially displaces the plug member such that fluid is allowed to pass through the at least one fluid flow window. Each plug member is configured and positioned so that when the wick cartridge is received into the vaporization chamber, the wick casing contacts the plug member and at least partially distorts the plug member such that fluid is allowed to pass through the at least one fluid flow window. Each plug member comprises a frangible portion configured and positioned so that when the wick cartridge is received into the vaporization chamber, the wick casing contacts and fractures the frangible portion so that fluid is allowed to pass through the at least one fluid flow window.

The method further comprises: removing the wick cartridge from the vaporization chamber, thereby returning the plug member to its initial configuration in which fluid flow through the fluid flow window is prevented.

Another aspect of the invention includes a personal vaporizer comprising: a main body; and a vaporization chamber within the main body.

Another aspect of the invention includes a personal vaporizer comprising: a wick cartridge.

Another aspect of the invention includes a method of operating a personal vaporizer, the method comprising: establishing fluid communication between a fluid reservoir and a wick body; allowing vaporizable liquid to flow to and through the wick body; and
powering a heating element to vaporize the vaporizable liquid.

### BRIEF DESCRIPTION OF THE DRAWINGS

The invention can be more fully understood by reading the following detailed description together with the accompanying drawing, in which like reference indicators are used to designate like elements, and in which:

FIG. 1 is a cross-sectional view of a personal vaporizer usable in conjunction with embodiments of the invention;

FIG. 2 is a perspective view of a composite wick according to an embodiment of the invention;

FIG. 3 is a perspective view of a composite wick according to an embodiment of the invention;

FIG. 4 is a perspective view of a composite wick according to an embodiment of the invention;

FIG. 5 is a perspective view of a composite wick according to an embodiment of the invention;

FIG. 6 is a perspective view of a composite wick according to an embodiment of the invention;

FIG. 7 is a perspective view of a composite wick according to an embodiment of the invention;

FIG. 8 is a perspective view of a composite wick according to an embodiment of the invention;

FIG. 9 is an end view of a composite wick according to an embodiment of the invention;

FIG. 10 is a perspective view of a portion of a personal vaporizer including a composite wick according to an embodiment of the invention;

FIG. 11 is a perspective view of an encased composite wick according to an embodiment of the invention;

FIG. 12 is a cross-sectional view of a personal vaporizer usable in conjunction with embodiments of the invention;

FIG. 13 is a perspective view of a composite wick according to an embodiment of the invention;

FIG. 14 is a section view of a vaporizer incorporating a composite wick according to an embodiment of the invention;

FIG. 15 is an enlarged section view of a portion of the vaporizer of FIG. 14;

FIGS. 16A and 16B are section views of a portion of a personal vaporizer incorporating a composite wick according to an embodiment of the invention;

FIGS. 17A and 17B are section views of a portion of a personal vaporizer incorporating a composite wick according to an embodiment of the invention;

FIGS. 18A and 18B are section views of a portion of a personal vaporizer incorporating a composite wick according to an embodiment of the invention;

FIGS. 19A and 19B are section views of a portion of a personal vaporizer incorporating a composite wick according to an embodiment of the invention;

FIGS. 20A-20E are section views of a composite wick cartridge and a portion of a personal vaporizer according to an embodiment of the invention; and

FIGS. 21A-21C are section views illustrating a portion of a personal vaporizer according to an embodiment of the invention.

### DETAILED DESCRIPTION OF THE INVENTION

While some micro-vaporizers use other mechanisms for transport of the vaporizable fluid from a reservoir to a vaporization chamber, most use some form of wick or fibrous wicking materials. In general, the wicking materials are selected based on their wicking properties (capillarity, porosity, hydrophilicity, surface energy, etc.), compatibility with the vaporizable fluid, and heat tolerance. In most cases, these materials are also likely to be selected so that they do not themselves contribute to the material exiting the vaporization chamber. In personal vaporizers, for example, wicking materials are chosen to impart as little or no flavor as possible.

The present invention provides composite micro-vaporizer wicks and wicking materials that provide, not only the requisite properties for transporting the vaporizable fluid, but also a mechanism for supplementing the active substances in the vaporized fluid. As will be discussed in more detail hereafter, the composite wick materials of the invention may be in the form of woven or non-woven fibrous materials in combination with embedded, trapped, adhered or alternately layered active additive materials. They are generally configured so that, in transport from the fluid reservoir to the vaporization chamber and/or heating element, the vaporizable fluid must come into contact with the active additive materials. Portions of the active additive materials may be released into the fluid or may otherwise affect or impart desired characteristics to the fluid.

While usable in any micro-vaporizer, the present invention is particularly suited to use in personal vaporizers. In personal vaporizers, wicking materials are chosen to impart as little or no flavor as possible, but additional materials are often added to the liquid to impart flavor. Recently, a new approach has been developed for flavor enhancement. In this approach, fluid transport structures provide, not only the requisite properties for transporting the vaporizable fluid, but also a mechanism for supplementing the active substances in the vaporized fluid. In particular, certain personal vaporizers use fluid transport structures that incorporate composite wicks that have a base wick structure and one or more active materials designed to release an agent into fluid that passes through the wick structure. (See, e.g., U.S. Pat. No. 10,792,443 (“the '443 Patent”), filed Jun. 30, 2017, the complete disclosure of which is incorporated herein by reference.)

The invention will be described in more detail using examples and embodiments geared primarily to personal vaporizers. It will be understood, however, that the methods of the invention are not limited to such applications and can be applied to any micro-vaporizer device. It will also be understood that, while many of the examples used herein refer to the use of composite wicks or other fluid transport systems incorporating active materials, the methods and devices of the invention may be used with any fluid transport system that uses a wick or wicking materials.

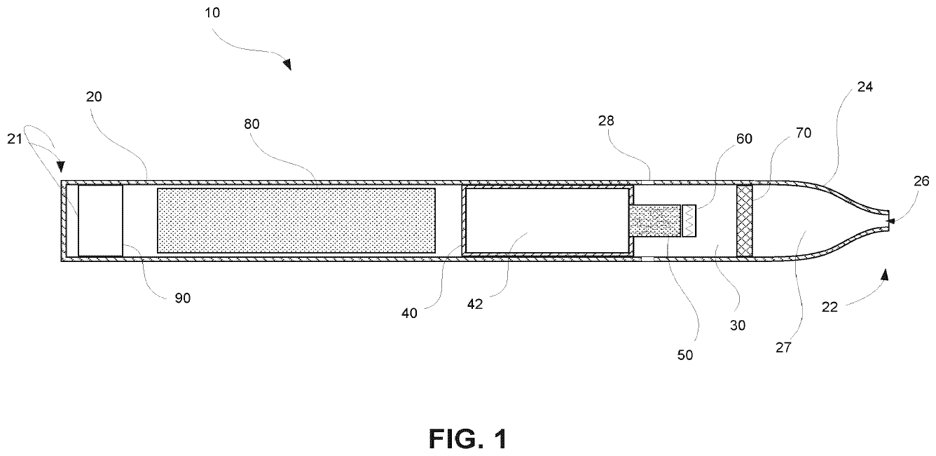

With reference to FIG. 1, a typical personal vaporizer 10 comprises a cylindrical casing 20 having a distal end 21 and a proximal end 22. At its proximal end 22, the casing 20 is formed into a mouthpiece 24 having a passage 26 providing fluid communication between the atmosphere and an exit chamber 27 inside the casing 20. The casing 20 also has one or more air holes 28 to allow air to flow from the atmosphere into a vaporization chamber 30 inside the casing 20 when a relative vacuum is applied at the mouthpiece passage 26 (e.g., by inhalation of a device user). The air drawn in through the air hole(s) 28 passes through a filter 70 which divides the vaporization chamber 30 and the exit chamber 27.

The personal vaporizer 10 further comprises a fluid reservoir 40 in which is disposed a vaporizable fluid 42. The fluid reservoir 40 may be configured as a simple tank in which the fluid 42 is disposed. In some embodiments, the reservoir 40 may be or include a housed or unhoused adsorptive or absorptive material or structure that retains the vaporizable fluid 42. A fluid transport structure 50 is configured and positioned to be in contact with the fluid 42 in the reservoir 40 and for drawing the fluid 42 out of the reservoir 40 and into the vaporization chamber 30. The fluid transport structure 50 may be further configured for bringing the drawn fluid 42 into close proximity or in contact with a heating element 60. The heating element 60 may be configured to heat the vaporizable fluid through any conductive, convective, and/or radiative heat transfer mechanism. In typical vaporizers, the heating element 60 is or includes a resistance element in the form of a wire coil. In some cases, the resistance element is housed within a heat conductive casing.

The illustrative personal vaporizer 10 also comprises a battery 80 for powering the heating element and a control unit 90. It will be understood that the configuration and relative positioning of the components of the personal vaporizer 10 may be widely varying and that additional components (e.g., an airflow controller for regulation of the amount of air flow through the holes 28) may be included.

To use the personal vaporizer 10, a user activates the heating element 60 and draws air through the device by inhaling through the mouthpiece. The vaporizable fluid 42 in the chamber 30 is heated to its vaporization point by the heating element 60. The resulting vapor mixes with air drawn through the air holes 28 and the mixture is drawn through the filter 70 and the exit chamber 27 and out through the mouthpiece passage 26.

The fluid transport structure 50 of the personal vaporizer 10 may be or comprise a wick or collection of wicking material. Typical personal vaporizer wicks are formed from organic fiber materials such as cotton, jute, flax, cellulose, or hemp. Some non-organic materials such as silica, carbon, and non-organic polymer fibers, ceramics and steel mesh may also be used. In general, vaporizer wicks can be formed from any material that is thermally stable and that provides sufficient wicking action to transport the vaporizable fluid 42 from the reservoir 40 to the heating element 60.

The composite wicks of the present invention are configured to provide stable, consistent wicking characteristics, but also provide the benefits of including one or more active materials that come into contact with the fluid prior to and/or during vaporization. As used herein, the term “active material” refers to any material that controllably alters or adds to the vaporization products of the device. Depending on the application, active materials can include, without limitation, plant material, minerals, deodorizing agents, fragrances, insect repellants, medications, and disinfectants and any material or structure containing or incorporating any of the foregoing.

In the specific instance of personal vaporizers, active materials may include flavorant substances that augment the flavorant of the vaporizable fluid. These may include, without limitation, marijuana, hemp, cannabidiol (cbd), citronella, geraniol, mint, thyme, tobacco, Salvia dorrii, Salvia, Passiflora incarnata, Arctostaphylos uva-ursi, Lobelia inflata, lemon grass, cedar wood, clove, cinnamon, coumarin, helio, vanilla, menthol, eucalyptus, peppermint, rosemary, lavender, licorice, and cocoa and any material or structure containing or incorporating any of the foregoing. One active material of particular interest for personal vaporizers is tobacco, which can be provided in the form of whole tobacco leaves, shredded tobacco leaves, crushed and dried tobacco flakes, slivers of dried tobacco leaves, and shavings from dried tobacco leaves. In some embodiments, it may be incorporated into woven or a non-woven fiber sheet with tobacco material weaved or embedded into the non-woven fiber sheet.

In some cases, active materials may be selected based on their tendency to release flavoring or other agents upon heating. Some materials may, for example, begin to decompose or off-gas upon reaching a certain temperature. For any particular such active material, the temperature at which the material begins to decompose or off-gas is referred to herein as the material's release temperature. For a combustible active material, temperatures falling between the material's release temperature and its combustion temperature are referred to herein as being in the material's release temperature range.

The following paragraphs describe composite wicks according to various embodiments of the invention.

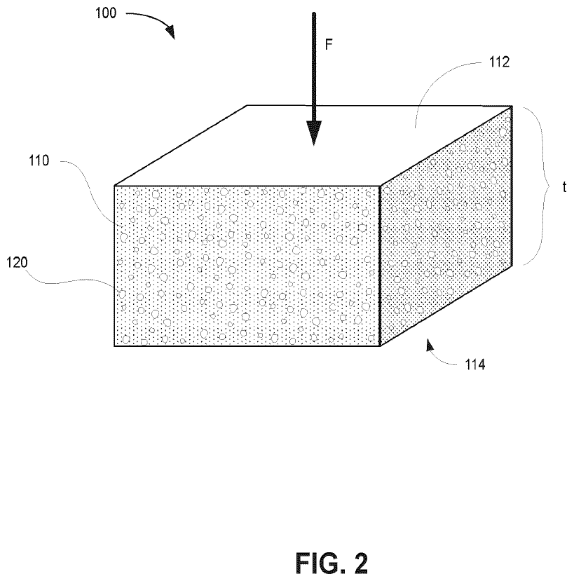

With reference to FIG. 2, a composite wick 100 according to an embodiment of the invention includes a base wick structure 110 and an inter-dispersed active material 120. The base wick structure 110 has an upstream surface 112 through which fluid is drawn into the wick 100 and a downstream surface 114. The primary flow direction through the wick 100 is designated by the arrow F. The distance between the upstream surface 112 and the downstream surface 114 defines a generally uniform thickness t. The downstream surface 114 will generally be oriented toward (i.e., facing) or in contact with a heating element to vaporize fluid at or near the downstream surface 114 or after the fluid has passed through the downstream surface 114.

The base wick structure may be formed from any of the wicking materials disclosed herein. In preferred embodiments, the base wick structure 110 is formed from organic or inorganic hydrophilic fibers. The particular fiber materials used may be selected according to the desired wicking and flow characteristics, compatibility with the vaporizable fluid and the active materials, and heat tolerance. In some cases, fibers can be coated with materials that increase hydrophilicity and/or surface energy to enhance the wicking action of the fiber. Wicking can also be optimized based on fiber size and type (e.g., stranded versus staple fibers).

In some applications, the wick material may also be selected, in part, based on its absorption and/or fluid retention characteristics. For example, the wick material may be selected to provide a particular range of saturation. In particular embodiments, the wick material of some or all of the base wick structure 110 may be configured specifically to optimize fluid retention. In such embodiments, the base wick structure may itself act as a reservoir for the vaporizable fluid. In this capacity, the base wick structure 110 may supplement or replace the separate micro-vaporizer reservoir. In micro-vaporizers having a fluid reservoir that is or includes an absorptive structure, the base wick structure 110 may be bonded to or integrally formed with the absorptive structure of the reservoir.

The wick material may also be selected based on the degree to which it expands upon contact with liquid. This can be used, inter alia, to provide a seal against liquid leakage and/or the passage of air through the wick structure.

The wick fibers may be woven or bonded to form a self-sustaining structure. In a particular embodiment, the wick structure 110 is a self-sustaining structure in which the fibers are thermally or chemically bonded to one another at spaced apart points of contact. Alternatively, the wick structure 110 may be formed as an unbonded, non-woven web. Such a web may be compressed or mechanically entangled (e.g., by needle punching) to impart a degree of structural integrity. Alternatively or in addition, structural integrity of the non-woven web may be maintained by enclosing the web in a casing or membrane or by bounding the web by other structural materials.

In all embodiments, the base wick structure 110 is formed with tortuous, interstitial passages that provide the desired capillarity and porosity characteristics of the wick. The structure of these passages can be tailored through material selection (e.g., fiber material, size, type, surface treatment, etc.) and selection of manufacturing methodology and process parameters. Flow characteristics may also be tailored through the use of fiber orientation. The flow characteristics of the base wick structure 110 may be tailored to provide optimum fluid flow rates. Such flow rates may be established based, not only on a desired vaporization rate, but on the desire to use the fluid to keep temperatures in the base wick structure 110 and the active material 120 within acceptable ranges. In some embodiments, for example, it may be desirable to tailor fluid flow so as to maintain the temperature of some or all of an active material within its release temperature range. In other embodiments, it may be desirable to tailor the fluid flow so that a limited, controlled amount of the active material exceeds its combustion temperature.

The active material 120 may be provided in any form that can be entrapped within or adjacent the interstitial passages of the base wick structure. Alternatively, the active material may be bonded to the base wick structure 110 that defines the interstitial passages. In various embodiments, the active material 120 can be provided in the form of powder, larger particles, or flakes. In embodiments in which the base wick structure 110 is formed from fiber materials, the active material 120 may be bonded to the fibers before or after formation of the base wick structure from the fibers. The active material 120 can be distributed randomly throughout the base wick structure 110 or can be preferentially distributed toward particular regions (e.g., with a higher density adjacent the upstream surface 112 or the downstream surface 114). As will be discussed in more detail below, the active material 120 may be disposed so that a portion of the material is exposed at one or both of the upstream and downstream surfaces 112, 114.

When placed in a micro-vaporizer device such as the personal vaporizer 10, the composite wick 100 is positioned so that the upstream surface 112 is in contact with the vaporizable fluid in or from a reservoir and the downstream surface 114 is adjacent or in contact with the heating element. The vaporizable fluid is drawn into and through the wick in flow direction F. As it passes through the wick, the fluid encounters and interacts with the active material 120 so that the nature and/or constitution of the fluid is changed. In particular embodiments, portions of the active material 120 may be dissolved in the fluid. In others, detached particles of the active material 120 may be suspended within the fluid. These particles may be small enough to pass through the tortuous passages in the wick structure 110 with the fluid. In some embodiments, flavoring material or other agents may be released into the vaporizable fluid as the result of the active material 120 being above its release temperature.

When the heating element of the micro-vaporizer device is activated, the fluid at or near the downstream surface 114 of the wick 100 is heated. Depending on the thermal output of the heating element, its proximity to the downstream surface 114 and the thermal characteristics of the base wick structure 110, the vaporizable fluid (as altered by the active material 120) may begin to vaporize within the base wick structure 110 so that the fluid exiting the wick is in vapor form or is a combination of liquid and vapor. Any fluid not vaporized within the wick 100 is vaporized after exiting the wick 100.

In addition to heating the vaporizable fluid, the heating element will also heat any portions of the base wick structure 110 and the active material 120 that are exposed at the downstream surface 114. Heat absorbed at the downstream surface 114 will also be conducted into the wick 100 establishing a temperature gradient therein. The vaporizable fluid flowing through the base wick structure 110, however, will absorb and carry away much of the heat taken in at the downstream surface 114. As noted, above, the flow characteristics of the base wick structure 110 can be tailored to provide a desired degree of cooling and, thus, a desired temperature gradient through the wick 100. This allows, for example, the ability to keep the temperature of the base wick structure 110 below material degradation temperatures. It also allows the ability to maintain at least some of the active material 120 within a desired temperature range (e.g., its release temperature range). This may be particularly significant for certain active material/vaporizable fluid combinations. In personal vaporizers, for example, certain organic active materials (e.g., tobacco and hemp) may not easily release a taste-affecting component into common e-liquid solutions such as propylene glycol (PG) and vegetable glycerine (VG) unless heated to within its release temperature range. Because the heating time interval during activation of such devices is relatively short (typically 2-3 seconds), it may be desirable to position active material within relatively close range of the heating element. Accordingly, in various composite wick embodiments, some or all of the active material may be positioned at or near the downstream surface of the wick. In general, it has been found that particularly satisfactory results may be achieved by configuring and positioning the composite wick so that at least some of the active material is within 5 mm of the heating element.

In some instances, the active material 120 used in the composite wick 100 may be provided in a form that has one or more dimensions that are larger than the remaining dimension(s). In such instances, the active material 120 may be positioned so as to have a particular orientation relative to the primary flow direction F through the wick structure 110.

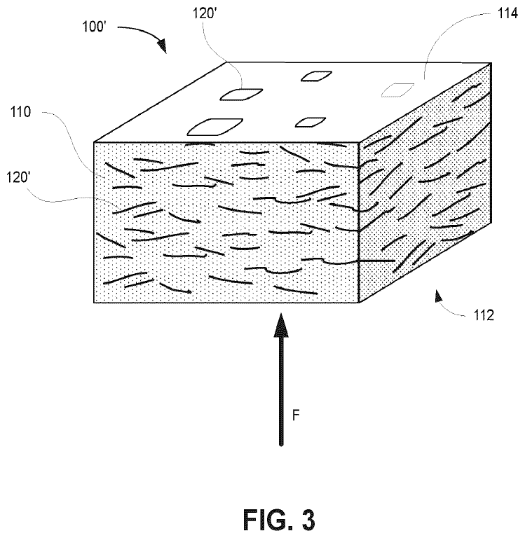

FIG. 3 illustrates a composite wick 100′ that is a particular variation of the composite wick 100 in which the active material 120′ is provided in the form of roughly planar flakes. As shown in FIG. 2, these flakes are positioned within the base wick structure 110 in such a manner so as to be roughly orthogonal to the primary flow direction F. In some embodiments, the active material 120′ may be porous so that when positioned orthogonal to the flow direction F, the wicked fluid may pass through as well as around the flakes of active material 120′, thereby increasing contact between the fluid and the active material 120′.

In some applications, it may be desirable for a portion of the active material to be exposed as the surface of one or both of the upstream and downstream surfaces 112, 114 of the composite wick 100. This provides direct exposure of some of the active material 120 to the heating element, which may be advantageous in terms of heating that portion of material above its release temperature. One example of this is where the active material in a personal vaporizer wick is tobacco. Heretofore, efforts to mimic the smoky, burning flavor of a cigarette or cigar in personal vaporizers have been largely unsuccessful. It has been found, however, that in certain embodiments of the composite wicks of the invention, tobacco materials can be disposed so that a portion of the tobacco is directly exposed to the heating element of the micro-vaporizer. This direct exposure results in the tobacco material being heated above its release temperature, which results in additional particles and/or gas products entering the vapor/air mixture in the vaporization chamber. As previously described, the flow characteristics of the base wick structure 110 can be tailored to assure that the fluid flowing around the tobacco material cools it enough to prevent the tobacco material from reaching its combustion temperature (i.e., to keep it from actually burning). In some cases, it may actually be desirable for a small amount of the tobacco to burn. In such cases, the base wick structure 110 can be tailored so that a controlled amount of the tobacco material reaches or exceeds its combustion temperature.

In the composite wick 100′ of FIG. 3, it can be seen that the flake-like active material 120′ (which could be, for example, tobacco flakes) is distributed in the base wick structure 110 in such a way that portions of some flakes are exposed at the downstream surface 114. These flakes are still held in place by the base wick structure 110, but will be directly exposed to the heating element when the wick 100 is positioned in the micro-vaporizer.

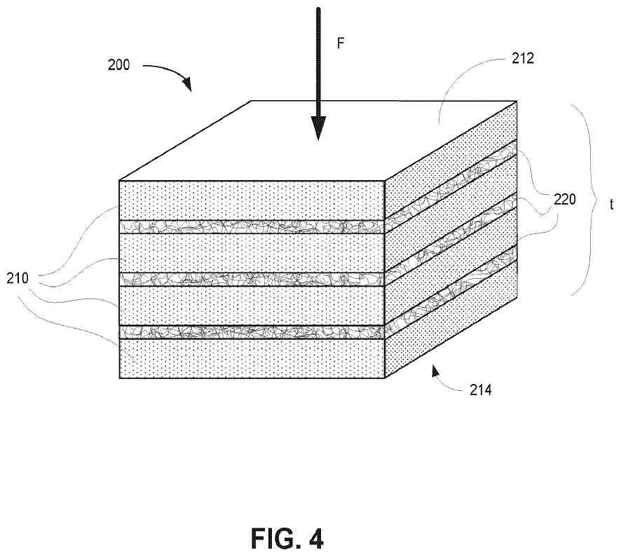

FIG. 4 illustrates a composite wick 200 according to another embodiment of the invention. The composite wick 200 is a layered structure that includes two or more base wick layers 210 alternating with one or more active material layers 220. The outermost base wick layers 210 define an upstream surface 212 through which fluid is drawn into the wick 200 and a downstream surface 214. The primary flow direction through the wick 200 is again designated by the arrow F. The distance between the upstream surface 212 and the downstream surface 214 defines a generally uniform thickness t. The downstream surface 214 will generally be oriented toward or in contact with a heating element to vaporize fluid at or near the downstream surface 214 or after the fluid has passed through the downstream surface 214. The base wick layers 210 may be formed from the same materials and have substantially the same structure as the base wick structure 110 of the composite wick 100. Thus, each base wick layer 210 is formed with tortuous, interstitial passages that provide the desired capillarity and porosity characteristics of the wick. It will be understood, however, that the characteristics of the layers 210 need not be the same. For example, different layers may have different thicknesses and/or flow-through or wicking properties. In some embodiments, the downstream-most base wick layer 210 may be configured to inhibit passage of liquid through the downstream surface 214 while allowing vaporization products to pass through unimpeded. This reduces leakage of liquid vaporization fluid past the heating element of the vaporization device.

Each active material layer 220 is a porous layer that is or includes an active material. The active material layer 220 may be a monolithic or self-sustaining structure bonded to or held in place by the surrounding base wick structures 210. Alternatively, the active material layer 220 may be a non-structural layer of unbonded particles, fibers, flakes or leaves. In a particular embodiment, the active material layer 220 may be a collection of flakes or whole or partial leaves (e.g., tobacco flakes or leaves) that are pressed together to provide a degree of structural integrity. In another particular embodiment, the active material layer 220 may be tobacco paper. In all cases, the active material layer 220 is porous (either as-formed or due to perforation) to allow fluid to flow through the layer. The lateral surfaces of the active material layers 220 (or the entire composite wick 200) may be surrounded by a casing or membrane to maintain structural integrity. Such a casing may be permeable or impermeable and may itself be or include an active material.

In some embodiments, the active material layers 220 may each be or include a wicking material in which an active material is disposed or to which an active material is bonded. The active material layers 220 could each, for example, be similar to the composite wick 100, providing both wicking and active material enhancement. In other embodiments, the base wick layers 210 may be similar to the composite wick 100 with active material dispersed therein.

In embodiments where there are multiple active material layers 220, the characteristics of each layer need not be the same. For example, different layers may have different active materials or may have different amounts of the same active material. They may also have different thicknesses and/or flow-through or wicking properties.

The placement and use of the composite wick 200 is substantially similar to that of the previously described composite wick 100. When placed in a micro-vaporizer device such as the personal vaporizer 10, the composite wick 200 is positioned so that the upstream surface 212 is in contact with the vaporizable fluid in or from a reservoir and the downstream surface 214 is adjacent or in contact with the heating element. The vaporizable fluid is drawn into and through the wick in flow direction F. The porous nature of the relatively thin active material layers 220 allows the passage of the fluid through these layers from base wick layer to base wick layer. In embodiments where the base wick layers 210 are formed from fiber materials, fluid flow across the active material layer 220 may be further enhanced by the fact that the boundaries between the active material layers 220 and the base wick layers 210 are not distinct. The region in the vicinity of such a boundary will actually contain both fiber material from the wick layer and material from the active material layer.

It will be understood that passage through the composite wick 200 necessarily requires fluid to pass through the active material layers 220. As in the previous embodiment, interaction with the active material results in the nature and/or constitution of the fluid changing as it passes through the wick.

As before, when the heating element of the micro-vaporizer device is activated, the a temperature gradient is established within the wick 200 and fluid at or near the downstream surface 214 of the wick 200 is heated to vaporization. The fluid flow characteristics of the base wick layers 210 and the active material layers 220 can be tailored to produce a desired temperature gradient.

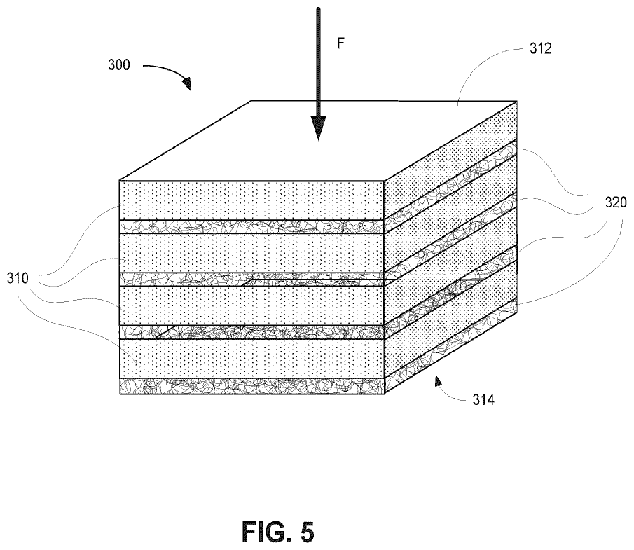

As noted above, the base wick layers 210 may be loaded with active material in a fashion similar to the composite wick 100. Such active material can be provided in such a form and disposed in such a way that a portion is exposed at the downstream surface 214 in a manner similar to that described for the composite wick 100′. This provides direct exposure of the active material to the heating element. With reference to FIG. 5, a composite wick 300 according to another embodiment of the invention provides significantly greater exposure of the active material to the heating element when the wick is installed in a micro-vaporizer. The wick 300 is similar to the previous wick 200 in that it has alternating base wick layers 310 and active material layers 320 and in that the upstream-most base wick layer 310 defines the upstream surface 312 of the wick 300. Further, the materials and configurations of the base wick layers 310 and the active material layers 320 are substantially similar to those of the composite wick 200. The composite wick 300 differs, however in that the downstream-most layer is an active material layer 320 that defines the downstream surface 314. This means that, in embodiments where the downstream-most active material layer 320 is formed entirely from an active material, the amount of active material surface area exposed to the heating element is maximized. As before, the fluid flow characteristics of the base wick layers 310 and the active material layers 320 can be tailored to produce a desired temperature gradient. For example, the flow characteristics may be established so as to maintain as much as possible of the active material in the active material layers 320 within the release temperature range of the active material.

While the direct presentation of an active material layer to the heating element as in the use of composite wick 300 may be advantageous for some micro-vaporizer applications (e.g., where the wick is for use in a personal vaporizer and the active material is tobacco), there may be some applications where it would be advantageous to have an exposed active material layer at the upstream surface of the composite wick. In such cases the configuration of the composite wick 300 could be reversed, with an active material layer at the upstream surface and a base wick layer at the downstream surface. In other embodiments, a layered composite wick could have active material layers at both upstream and downstream surfaces.

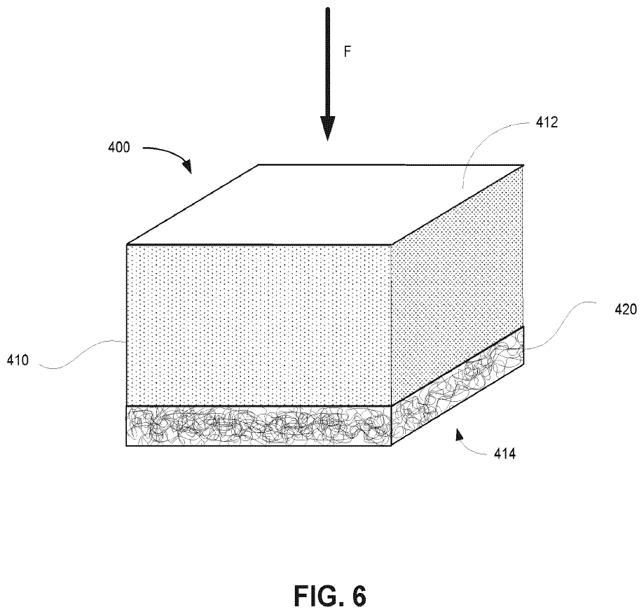

With reference to FIG. 6, a composite wick 400 according to yet another embodiment of the invention has a base wick main body 410 defining an upstream surface 412 and an active material layer 420 bonded to or held in abutment with the base wick main body 410 downstream side of the main body 410. The wick main body 410 can have similar materials and configuration to the base wick 110 of the composite wick 100. It can also have active material attached to or disposed within its tortuous passages. The active material layer 420 can be substantially similar to the active material layers of the previously described layered composite wicks 200, 300. Placement of the active material layer 420 at the downstream surface 414 provides similar exposure advantages to those of the composite wick 300 and the fluid and/or vaporization products must pass through the porous active material layer prior to exiting the wick 400 and entering the vaporization chamber. As before, the fluid flow characteristics of the base wick main body 410 and the active material layer 420 can be tailored to produce a desired temperature gradient. For example, the flow characteristics may be established so as to maintain as much as possible of the active material layer 420 within the release temperature range of the active material.

In the wick 400 illustrated in FIG. 6, the active material layer 420 and the downstream surface 414 are positioned opposite the upstream surface 412. It will be understood, however, that the downstream surface could be or include one or more of the lateral surfaces of the wick. In such cases, an active material layer may be positioned on any one or more of the lateral surfaces in addition to or instead of the surface opposite the upstream surface 412. In a particular embodiment, a base wick main body 410 could have an active material layer on each lateral side of the wick (or around the complete circumference if the wick main body 410 is circular). Such laterally-oriented active material layers could also be added to any of the composite wicks described herein.

As was the case with the composite wick 300 configuration, it would also be possible to reverse the position of the active material layer, placing it on the upstream side of the base wick main body.

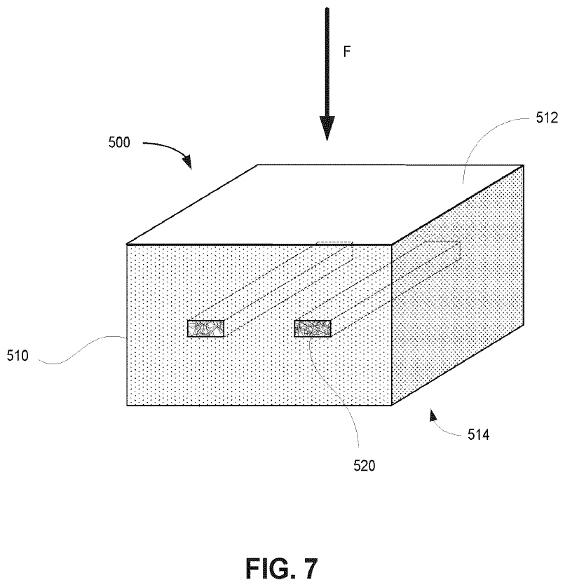

With reference to FIG. 7, a composite wick 500 according to yet another embodiment of the invention has a base wick main body 510 defining an upstream surface 512 and a downstream surface 514. The wick main body 510 can have similar materials and configuration to the base wick 110 of the composite wick 100. It can also have active material attached to or disposed within its tortuous passages. The composite wick 500 also has one or more elongate active material bodies 520 embedded within the wick main body. The active material bodies 520 may be in the form of slivers or rods of active material or can be substantially similar to the active material layers of the previously described layered composite wicks 200, 300, but with limited lateral extent. The active material bodies 520 are oriented generally orthogonal to the flow direction F so that wicked fluid can flow around and/or through the active material to maximize interaction between the fluid and the active material. The fluid flow characteristics of the base wick main body 510 can be tailored to produce a desired temperature gradient within the wick 500.

The base wick structures of the above-described composite wick embodiments may have any lateral cross-section including, but not limited to, rectangular (as shown in the figures) or other polygonal shape, circular, elliptical, or free-form. The upstream and downstream surfaces 112, 114 may be substantially planar as shown or may have a degree of uniform curvature or other desired topography.

The descriptions and illustrations of the foregoing embodiments are presented in the context of a generally planar wick structure with limited lateral extent. It will be understood, however, that any of the foregoing embodiments may be used to provide a generally planar wick sheet having relatively large lateral dimensions relative to the thickness of the wick structure.

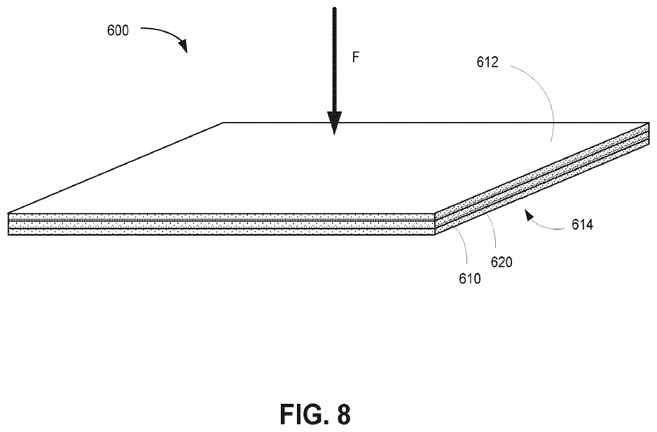

FIG. 8 illustrates an exemplary composite wick 600 that is in the form of a generally planar sheet. The composite wick 600 is similar in form to the layered composite wick 200 of FIG. 4 with similar base wick layers 610 and active material layers 620. In this embodiment, however, the upstream surface 612 and downstream surface 614 present large areas relative to the cross-sectional are of the wick. This provides for a much higher available area for contact with fluid in the reservoir of a micro-vaporizer and/or for exposure to the micro-vaporizers heating element.

Depending on the wick and active materials and the desired layup of the wick, a composite wick sheet may be formed through weaving, bonding, pressing, or calendering. In some embodiments, the wick sheet may be formed by producing (e.g., by melt-blowing, spun bonding, melt-spinning, or a combination thereof) a loose fiber web which is then passed through a die, pressed or calendered to form a close-packed sheet of fiber. Individual layers of active material can be similarly formed by weaving, bonding, pressing or calendering and then bonded to or pressed with base wick layers to form layered composite wicks. In embodiments where the active material is distributed throughout the base wick material, the active material can be deposited into or bonded to the wick material (e.g., organic or inorganic fibers) prior to the wick material being woven, bonded, pressed, or calendered into a sheet. Sheets may be formed using both batch and continuous manufacturing processes.

The resulting composite wick sheet may then be cut to desired dimensions. The sheet may be cut to produce relatively large sheets like the composite wick 600 or it may be cut to provide multiple composite wicks of smaller dimensions like those shown in FIGS. 2-7. Typical lateral dimensions for a rectangular cross-section composite wick for use in personal vaporizers would be 0.25-1.25 cm by 0.5-2.0 cm. A generally suitable thickness would typically be in a range of 0.1-0.5 cm. A particularly suitable thickness would be in a range of 0.15-0.45 cm and, even more suitably, in a range of 0.25-0.35 cm.

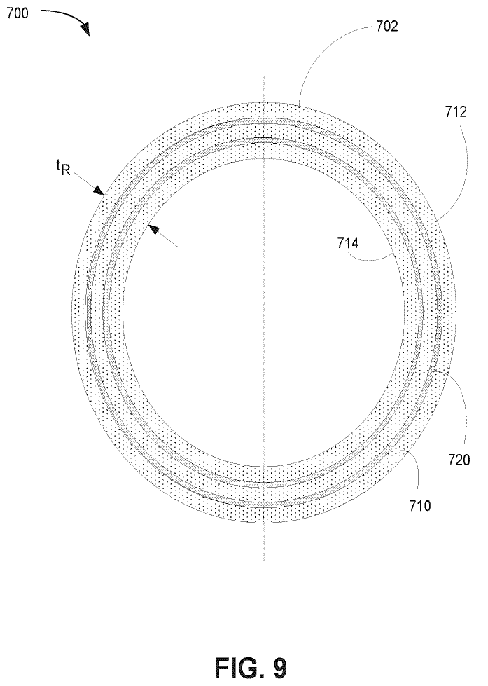

In another embodiment illustrated in FIG. 9, a composite wick 700 according to an embodiment of the invention is formed into an annular cylindrical body 702 having an outer surface 712 and an inner surface 714. The composite wick 700 is configured so that the primary flow direction for wicked fluid is radially through the radial thickness t_{R} of the body 702.

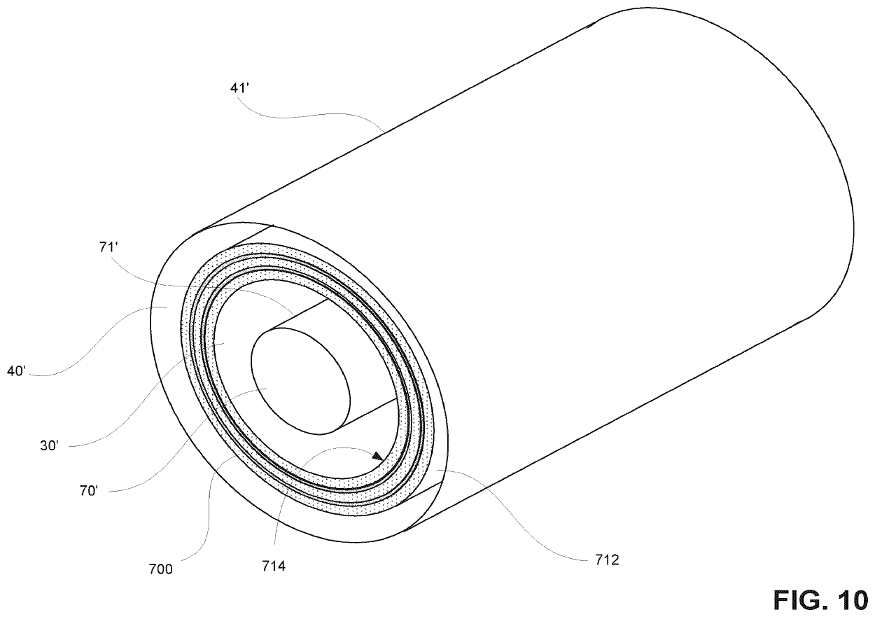

Depending on the configuration of the micro-vaporizer in which the composite wick 700 is to be used, the primary flow direction may be inward or outward. For example, as shown schematically in FIG. 10, a micro-vaporizer may have an annular cylindrical vaporizable fluid reservoir 40′ with an outer wall 41′ and a centrally located cylindrical heating element 70′. In this exemplary application, the composite wick 700 is placed radially intermediate the reservoir 40′ and the heating element 70′ so that the outer surface 712 provides an inner boundary for the reservoir 40′ and is in contact with fluid in the reservoir40′. The inner boundary 714 of the composite wick 700 is exposed to the outer surface 71′ of the heating element 70′. In this configuration, the primary flow direction through the composite wick 700 will be radially inward. With the vaporization products entering the vaporization chamber 30′ surrounding the heating element 70′.

In some embodiments, the inside diameter of the composite wick 700 may be sized to be very close to or even in contact with some or all of the heating element surface 71′. It will be understood that in some instances, the heating element 70′ could be configured in forms other than a smooth cylinder so that some areas of the wick inner surface 714 may be closer in proximity to (or in contact with) the heating element 70′ than other areas.

As shown in FIGS. 9 and 10, the composite wick 700 is a layered structure similar to those of the wicks 200, 600 of FIGS. 4 and 8, having a plurality of base wick layers 710 and active material layers 720. It will be understood, however, that similar cylindrical wicks may be formed from any of the previously described composite wick structures. Regardless of their cross-sectional form, any of the annular cylindrical wicks of the invention can initially be formed as sheet-like composite wicks (like the composite wick 600 of FIG. 8), which are than curved into an annular cylinder. This may be accomplished using a cylindrical mandrel or, in some embodiments, directly on a cylindrical casing of the heating element 70′.

Regardless of which surface 712, 714 is the downstream surface, the fluid flow characteristics of the base wick material and, in some cases, the active material used to form the wick 700 can be tailored to produce a desired temperature gradient within the wick adjacent the downstream surface.

In a variation of the previously described embodiment, a composite cylindrical wick 700 may also be adapted for use in a micro-vaporizer having an annular heating element that would at least partially surround the cylindrical wick 700. In such an embodiment, the composite wick 700 would be configured to draw fluid from a central reservoir source toward the outer surface 712, which would be exposed to the heating element. In such an embodiment, the cylindrical wick 700 could be sized for insertion into the annular passage of the heating element.

It will be understood that in any of the composite wicks of the invention, surface areas (e.g., the lateral surface areas of the wicks shown in FIGS. 2-8 or the end areas of the cylindrical wick of FIG. 9) through which fluid flow is not desired may be sealed using a casing or wall. In some instances, flow through such surface areas may be prevented by a wall of the micro-vaporizer structure in which the wick is installed.

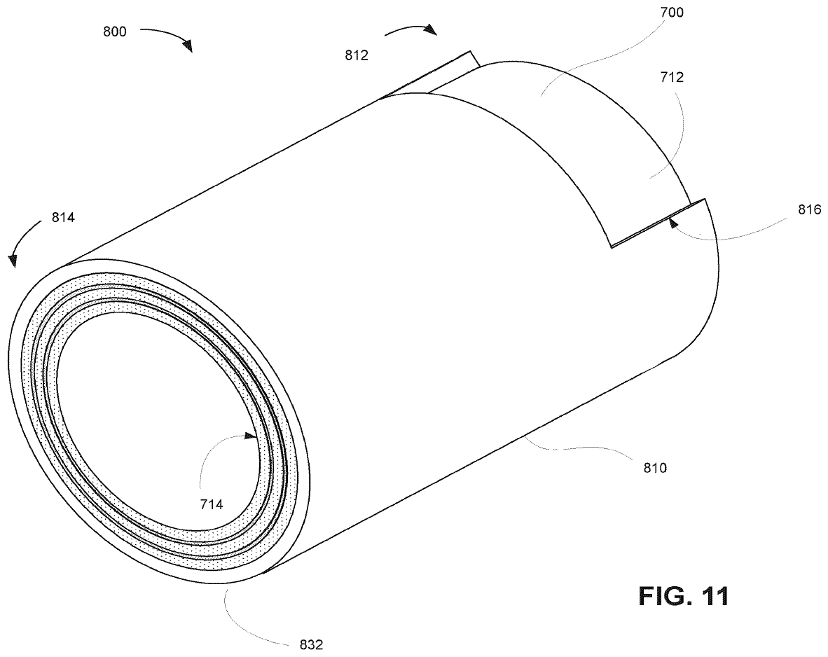

In some applications, it may be advantageous to have a large area of exposure of the downstream surface of the wick to the heating element of a micro-vaporizer, but a relatively small area of exposure of the upstream surface of the wick to the fluid in the micro-vaporizer's fluid reservoir. In such applications, a portion of the upstream wick surface may be shielded from the fluid reservoir by a wall, casing or membrane. FIG. 11 illustrates an embodiment of the invention in which the cylindrical annular composite wick 700 is surrounded by a casing 810 to form a cartridge 800 extending from a first cartridge end 812 to a second cartridge end 814. The cylindrical casing 810 is formed with a notch 816 at the first cartridge end that exposes a portion of the outer surface 712 of the cylindrical composite wick 700. When positioned in a micro-vaporizer having the reservoir and heating element configuration of FIG. 10, the casing 810 provides a wall between the reservoir 40′ and the outer surface 712 of the cylindrical composite wick 700. In use, fluid from the reservoir 40′ will be drawn into the wick 700 only through the surface area exposed by the notch 816. The fluid will be drawn both inwardly toward the heating element 70′ and longitudinally toward the second cartridge end. The structure of the base wick portion of the composite wick 700 may be configured so as to enhance the longitudinal flow characteristics of the wick so as to assure that vaporizable fluid reaches the inner surface 714 along the entire length of the cylindrical wick 700.

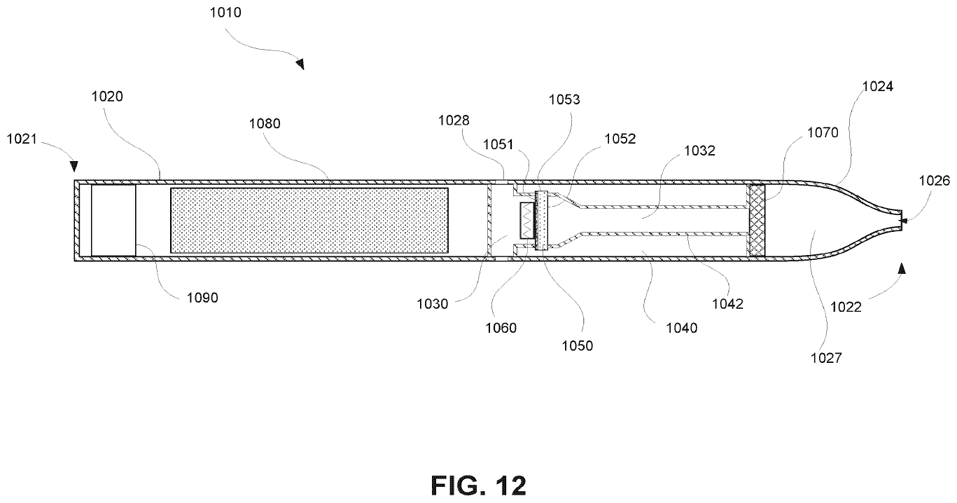

The composite wicks of the invention may be adapted to virtually any reservoir/heating element configuration. FIG. 12, for example, illustrates a personal vaporizer 1010 similar to the personal vaporizer of FIG. 1 in that it has a cylindrical casing 1020 with a distal end 1021 and a proximal end 1022, a mouthpiece 1024 with an exit passage 1026, a filter 1070, and an exit chamber 1027. The personal vaporizer 1010 also has a battery 1080 and, optionally, a control unit 1090. Like the personal vaporizer 10 of FIG. 1, the personal vaporizer 1010 has one or more air holes 1028 through the casing 1020 to allow air to flow into a vaporization chamber 1030. It differs, however, in that it has a cylindrical fluid reservoir 1040 that surrounds a portion of the vaporization chamber 1030 and the heating element 1060. The heating element 1060 may advantageously be, for example, a coil or circular mesh resistance element. The heating element 1060 is positioned at or near the proximal end of the vaporization chamber 1030, which is in fluid communication with a chimney 1032 bounded by the inner wall 1042 of the reservoir. The chimney 1032 provides a conduit through which air and vaporization products pass from the vaporization chamber 1030 to the filter 1070 and exit chamber 1027.

To supply fluid for vaporization by the heating element 1060, the personal vaporizer 1010 is provided with a disc-like wick 1050 having distal, proximal and circumferential surfaces 1051, 1052, 1053. The wick 1050 is centered on the longitudinal axis of the personal vaporizer 1010 so that the wick's distal surface 1051 is adjacent or in contact with the heating element 1060. The wick 1050 is sized so that it extends outward to and through a circumferential opening 1041 in the inner wall 1042 of the fluid reservoir 1040. The wick 1050 is configured so that fluid in the reservoir 1040 is drawn into the wick 1050 through the circumferential surface 1056 and/or through portions of the distal and proximal surfaces 1051, 1052 adjacent the circumferential surface 1056. The wick 1050 is further configured so that the vaporizable fluid is drawn inwardly toward the longitudinal axis of the personal vaporizer 1010 and proximally toward the proximal surface 1052 where it is exposed to heat from the heating element 1060 and vaporized.

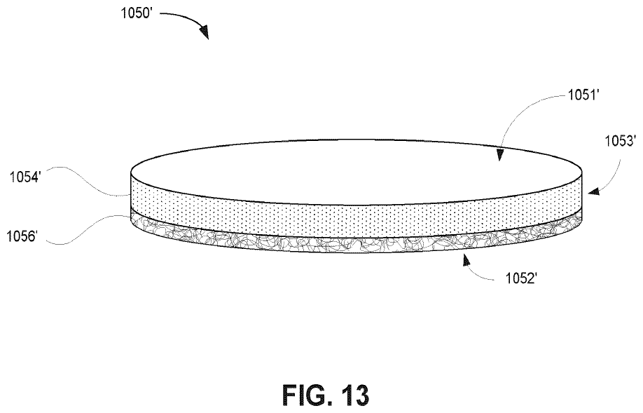

Any of the composite wick configurations discussed above can be used in the disc-like wick 1050 of the personal vaporizer 1010. FIG. 13 illustrates an exemplary composite wick 1050′ having similar characteristics to the wick 400 of FIG. 6. The composite wick 1050′ has a base wick main body 1054′ that defines the proximal surface 1052′ and an active material layer 1056′ bonded to or held in abutment with the base wick main body 1054′ that defines the distal surface 1051′. As previously described, such a configuration positions the active material for direct exposure to or contact with the heating element 1060. The wick main body 1054′ and the active material layer 1056′ are both formed as thin (i.e., disc-like) cylindrical elements and collectively define the circumferential surface 1053′.

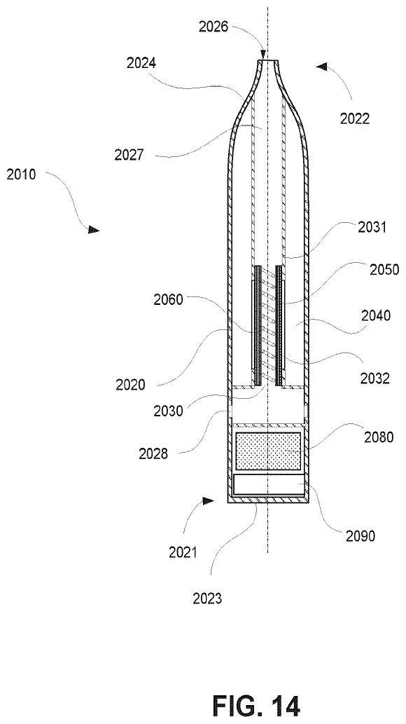

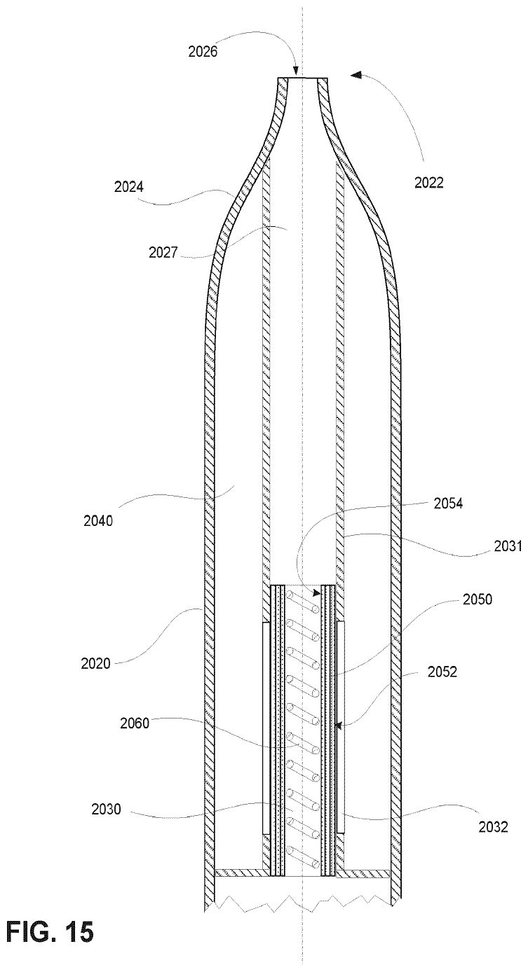

FIGS. 14 and 15 schematically illustrate a personal vaporizer 2010 according to an embodiment of the invention that has a relatively compact structure and may be configured to stand upright when not in use. The personal vaporizer 2010 has a cylindrical casing 2020 with a distal end 2021 and a proximal end 2022, a mouthpiece 2024 with an exit passage 2026, and an exit chamber 2027. A base member 2023 may be configured to act as a stand for placement of the vaporizer 2010 on a horizontal surface in the upright orientation shown in FIG. 14. The personal vaporizer 2010 also has a battery 2080 and, optionally, a control unit 2090. Like the personal vaporizer 20 of FIG. 1, the personal vaporizer 2010 has one or more air holes 2028 through the casing 2020 to allow air to flow into a vaporization chamber 2030.

It differs, however, in that it has a cylindrical fluid reservoir 2040 that surrounds the vaporization chamber 2030. A fluid flow window 2032 is provided through the wall 2031 of the vaporization chamber 2030 to allow vaporization fluid in the reservoir 2040 to flow therethrough. The fluid reservoir 2040 may be configured as a simple tank in which a vaporizable liquid 2042 is disposed (or disposable). In some embodiments, the reservoir 2040 may be or include a housed or unhoused adsorptive or absorptive material or structure that retains the vaporizable fluid 2042. A fluid transport structure 2050 is configured and positioned to be in contact with the fluid 2042 through the fluid flow window 2042 and for drawing the fluid 2042 out of the reservoir 2040 and into the vaporization chamber 2030. The fluid transport structure 2050 may be further configured for bringing the drawn fluid 2042 into close proximity or in contact with a heating element 2060. The heating element 2060 may be configured to heat the vaporizable fluid through any conductive, convective, and/or radiative heat transfer mechanism. In typical vaporizers, the heating element 2060 is or includes a resistance element in the form of a wire coil. In some vaporizers, however, the resistance element may be a flat plate or a three dimensional that presents a planar or curved heating surface. In some cases, the resistance element is housed within a heat conductive casing.

The vaporizer 2010 has an annular, cylindrical composite wick 2050 that may be positioned so that at least a portion of its outer circumferential surface 2052 is in registration with the fluid flow window 2032. In some embodiments, the wick 2050 may be sized and positioned so that it extends into the window 2032. In some embodiments, the wick 2050 may be sized and positioned so that it extends through the window 2032 into the reservoir 2040.

A coil-type heating element 2060 is disposed within the interior of the cylindrical composite wick 2050 and is connected via circuitry (not shown) to the battery 2080. The wick 2050 and heating element 2060 may be collectively sized and positioned so that the inner surface 2054 of the composite wick 2050 is closely adjacent or in contact with the outer surface of the heating element 2060. The wick 2050 may be substantially similar to the layered cylindrical wick 700 of FIG. 9. Alternatively, the wick 2050 may be formed as a single layer composite in which a base wick material has an active material dispersed there-within similar to what is illustrated in FIGS. 2 and 3. Regardless of the specific configuration of the wick body, the outer surface 2052 of cylindrical annular wick 2050 operates as an upstream surface through which vaporizable liquid can be drawn into the wick 2050 from the reservoir 2040 via the fluid window 2032. The fluid passes radially inward through the wick 2050 to the inner (downstream) surface 2054 where it is heated and vaporized by the heating element 2060. As the liquid passes through the wick 2050, it interacts with the active material that is interspersed within the base wick material and/or formed into layers alternating with layers of the base wick material.

As previously noted, the various composite wick embodiments described above can be used in any micro-vaporizer requiring transport of vaporizable fluid from a reservoir to another location where it is heated to vaporization. The wicks of the invention are particularly suited, however to use in personal vaporizers. Many users turn to these devices as alternatives to or replacements for burning tobacco products such as cigarettes, cigars, pipes, etc. For such users, the ideal replacement would be one that mimics the burning tobacco experience to the greatest extent.

Heretofore, personal vaporizers have been limited in their ability to mimic the burning tobacco experience. The typical vaporizable fluid used in these devices may include nicotine and a flavorant intended to mimic the taste of a tobacco product, but it does not actually include tobacco. The composite wicks of the invention provide the ability to impart tobacco characteristics to the vaporizable fluid and to even provide the ability to mimic the smoky burning sensation of a cigarette or cigar. This is accomplished by using real tobacco as the active material in the wick used to transport the vaporizable fluid. In this approach, the wick acts as both a liquid transport device and a flavoring agent. As has been discussed, the wicks may also provide a mechanism for directly exposing portions of tobacco material to the heating element, which produces a small degree of burning, the products of which are mixed with the vaporization products from the liquid. The combined products are then mixed with the air being drawn through the device and inhaled by the user.

The composite wicks of the invention provide a combination of desirable wicking properties typically imparted by base wick materials and fluid property enhancement typically provided by incorporated active materials. As has been discussed, the composite wicks may be configured in many different ways and additional components (e.g., an airflow controller for regulation of the amount of air flow through the holes 2028) may be included depending on the application and the configuration of the vaporizing device. In general, the composite wicks are positioned or positionable so as to be in fluid communication with the vaporizable fluid and are configured so as to draw the vaporizable fluid toward at heat source.

To use the personal vaporizer 10, a user activates the heating element 2060 and draws air through the device by inhaling through the mouthpiece 2024. The liquid 2042 at the inner circumferential surface 2054 and some or all of the vaporizable fluid 2042 in the fluid transport structure 2050 is heated to the liquid's vaporization point by the heating element 2060. The resulting vapor mixes with air drawn through the air holes 2028 and the mixture is drawn through the filter 2070 and the exit chamber 2027 and out through the mouthpiece passage 2026.

As previously noted, the fluid transport structure 2050 of the personal vaporizer 2010 may be or comprise a wick or collection of wicking material. Typical personal vaporizer wicks are formed from organic fiber materials such as cotton, jute, flax, cellulose, or hemp. Some non-organic materials such as silica, carbon, and non-organic polymer fibers, ceramics and steel mesh may also be used. In general, vaporizer wicks can be formed from any material that is thermally stable and that provides sufficient wicking action to transport the vaporizable fluid 2042 from the reservoir 2040 to the heating element 2060.

Also as previously noted, the fluid transport structure 2050 may comprise a composite wick, which may include a base wicking material and an active material. As described in the '443 Patent, active material may be dispersed through a fibrous wick body in some composite wick configurations. In other configurations, the wick body may be formed as a series of alternating layers of wicking material and active material. In some cases, active materials may be selected based on their tendency to release flavoring or other agents upon heating. Some materials may, for example, begin to decompose or off-gas upon reaching a certain temperature. For any particular such active material, the temperature at which the material begins to decompose or off-gas is referred to herein as the material's release temperature. For a combustible active material, temperatures falling between the material's release temperature and its combustion temperature are referred to herein as being in the material's release temperature range.

The inventors have found, however, that there are instances where prolonged exposure of composite wick components to a vaporizable fluid may produce undesirable effects. In some instances, for example, prolonged exposure or immersion of an active material in a vaporizable liquid can result in portions of the activated material decomposing or dissolving into the vaporizable liquid. In other instances, undesirable substances within the active material may be leeched out of the active material and into the vaporizable liquid.

In other instances, exposure of some active materials to vaporizable fluids may cause these materials to swell, which can have the effect of reducing the effective porosity and/or overall wicking capability of the composite structure.

In prior art vaporizers, the wicks or wicking materials used to transport liquid from a reservoir to a vaporization chamber contact and draw fluid as soon as liquid is introduced into the reservoir. This can result in long-term exposure of the fluid transport structure prior to first use, particularly in products that are packaged with liquid already loaded in the reservoir. This can present a significant issue for transport structures that incorporate active materials affected by liquid contact.

In order to avoid the effects of prolonged fluid exposure, it may be desirable to isolate the composite wick from the vaporizable fluid until just before use. The present invention contemplates several approaches to maintaining isolation until just before use. These approaches may include, without limitation, (1) providing a removable physical barrier between the wick and the vaporizable fluid reservoir, (2) providing a membrane or other fluid impervious barrier that can be selectively breeched to allow passage of the vaporizable fluid into communication with the wick, and (3) providing multi-component vaporizer structures configured to be joined or manipulated relative to one another to bring an otherwise isolated wick body into communication with vaporizable liquid. In each of these approaches, fluid communication between the wick and the vaporizable fluid can be selectively established by the user just prior to use. In each of these approaches, the vaporizer has an initial pre-use configuration in which the wick or other fluid transport structure is isolated from the liquid in the vaporizer's fluid reservoir. In embodiments that use a barrier, the pre-use configuration is typically one in which the barrier is intact and in place to prevent liquid from flowing out of the reservoir. In such embodiments, the vaporizer may be placed in the operational configuration by breeching, displacing, deflecting, or removing the barrier and (in some cases) placing the wick in position to receive liquid from the reservoir. This is accomplished by an action of the user, typically just prior to use.

The inventors have determined that concerns with long-term exposure prior to use can be reduced by preventing the fluid transport structure of a vaporizer from contacting the vaporizable liquid in the reservoir. The present invention provides vaporizers having a fluid transport structure that is isolated from the liquid in the fluid reservoir until a user selectively causes the transport structure to make contact and begin transporting the liquid. The invention encompasses several methods and mechanisms for isolating the fluid transport structure from the liquid and for selectively deisolating the structure (i.e., establishing liquid contact and flow through the structure). Deisolation by the user, will typically, but not necessarily, be accomplished just prior to first use of the vaporizer device. In many embodiments, deisolation is permanent, but in some embodiments it may be possible to reisolate the fluid transport structure from the liquid remaining in the reservoir.

Several examples of these approaches to wick isolation will now be provided in the context of a vaporizer having a similar structure and components to those of the vaporizer 2010 of FIGS. 14 and 15.

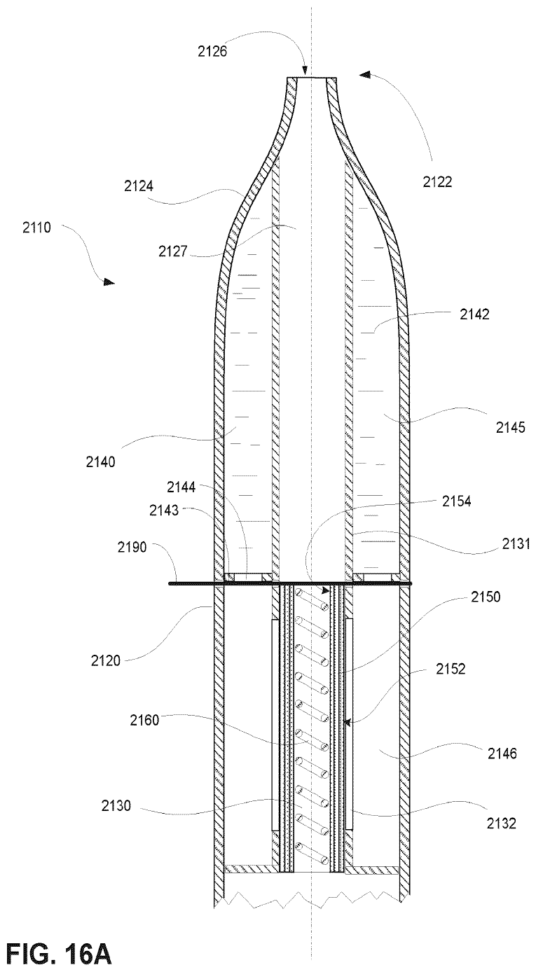

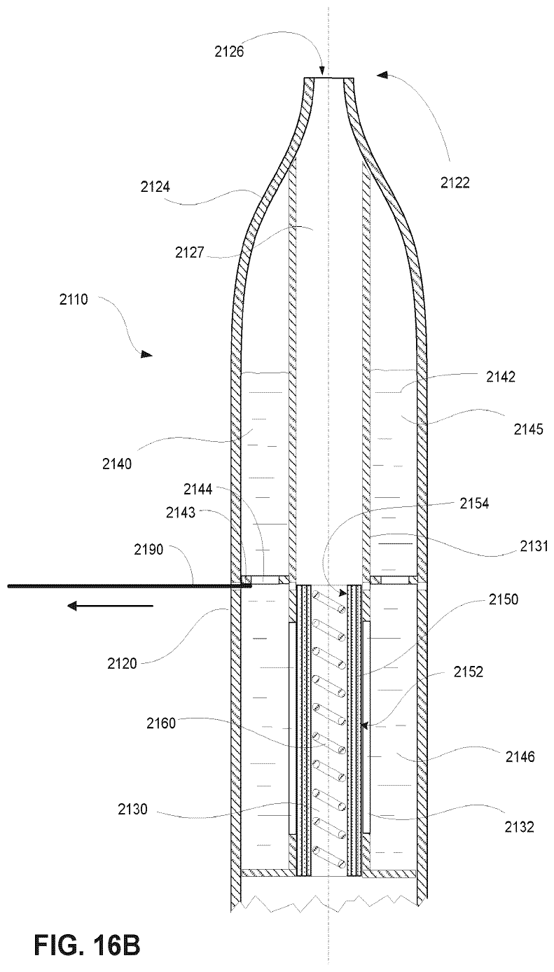

FIGS. 16A and 16B illustrate a portion of a vaporizer 2110 according to an embodiment of the invention having a cylindrical casing 2120 with a distal end 2121 and a proximal end (not shown), a mouthpiece 2124 with an exit passage 2126, and an exit chamber 2127. The personal vaporizer 2110 has a cylindrical fluid reservoir 2140 that surrounds the vaporization chamber 2130. The reservoir 2140 is divided into an upper reservoir chamber 2145 and a lower reservoir chamber 2146 by removable, fluid-impermeable barrier member 2190.

The barrier member 2190 may be a rigid planar structure or may be a pliable membrane. In some embodiments, the barrier member 2190 may extend across the entire interior of the cylindrical case 2120. In other embodiments, the barrier member 2190 may have a center perforation sized so that the barrier member only extends across the annular reservoir 140. In either case, the barrier member 2190 has a grasping portion 2192 extending through the case wall 2121 so that it can be grasped and removed to allow vaporizable fluid to flow from the upper chamber 2145 to the lower chamber 2146. A fluid flow window 2132 is provided through the wall 2131 of the vaporization chamber 2130 to allow vaporization fluid in the reservoir 2140 to flow therethrough. The fluid flow window 2132 is positioned below the barrier member 2190.

The vaporizer 2110 has an annular, cylindrical composite wick 2150 that may be positioned so that at least a portion of its outer circumferential surface 2152 is in registration with the fluid flow window 2132. In particular embodiments, the wick 2150 may be a composite wick having one or more active material layers or having an active material dispersed therein. In some embodiments, the wick 2150 may be sized and positioned so that it extends into the window 2132. In some embodiments, the wick 2150 may be sized and positioned so that it extends through the window 2132 into the lower chamber 2146 of the reservoir 2140.

As in the vaporizer 2010 of FIG. 14, a coil-type heating element 2160 is disposed within the interior of the cylindrical composite wick 2150. The wick 2150 and heating element 2160 may be collectively sized and positioned so that the inner surface 2154 of the composite wick 2150 is closely adjacent or in contact with the outer surface of the heating element 2160.

Prior to first use, the vaporizer 2110 may be as shown in FIG. 16A when in the vertical orientation. In this pre-use configuration, the impermeable barrier 2190 is in position and prevents vaporizable fluid 2142 from passing into the lower chamber 2146. Accordingly, there is no communication of the fluid 2142 with the composite wick 2150. In the operational configuration shown in FIG. 16B, the barrier 2190 has been removed, allowing the vaporizable liquid 2142 to drop into the lower chamber 2146 where it passes through the window 2132 to contact the upstream surface 2152 of the wick 2150. The vaporizer 2110 may now operate in a substantially similar manner to the vaporizer 2010 of FIGS. 14 and 15.

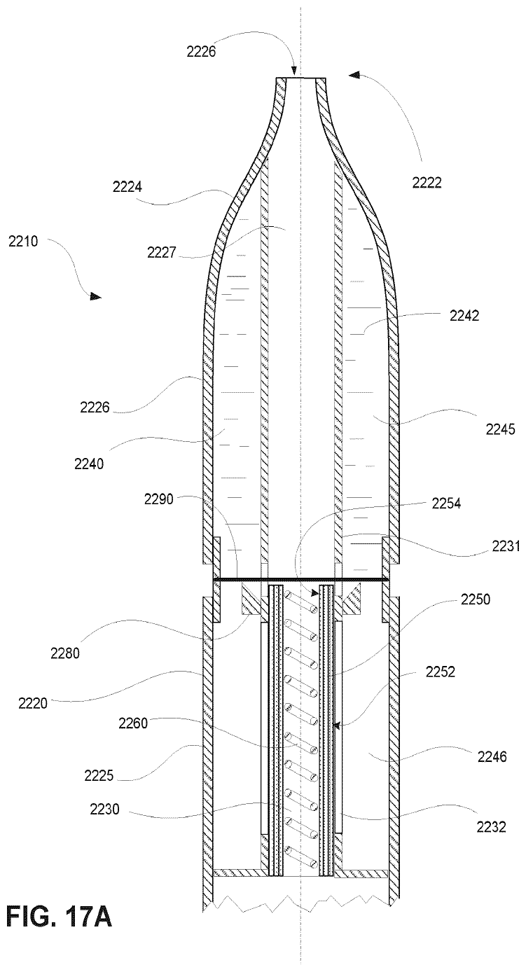

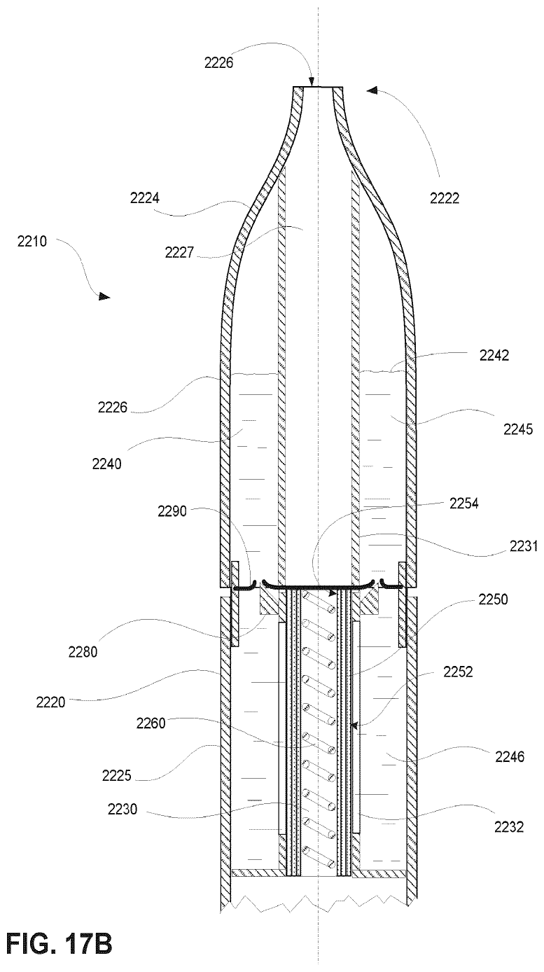

FIGS. 17A and 17B illustrate a portion of a vaporizer 2210 according to an embodiment of the invention having a cylindrical casing 2220 formed from an upper casing portion 2226 and a lower casing portion 2225. The upper casing portion 2226 has a distal end 2221 and a mouthpiece 2224 with an exit passage 2226, and an exit chamber 2227. The personal vaporizer 2210 has a cylindrical fluid reservoir 2240 that surrounds the vaporization chamber 2230. The reservoir 2240 is divided into an upper reservoir chamber 2245 positioned within the upper casing portion 2226 and a lower reservoir chamber 2246 positioned within the lower casing portion 2225. Prior to use, the upper and lower reservoir chambers 2245, 2246 are separated by fluid-impermeable membrane 2290. In some embodiments, the membrane 2290 may extend across the entire interior of the cylindrical case 2220. In other embodiments, the membrane 2290 may have a center perforation sized so that the barrier member only extends across the annular reservoir 2240. The membrane 2290 is configured so that it can be penetrated and/or torn when engaged by sharp contact members 2280 attached to the lower vaporizer structure.

The upper and lower casing portions 2226, 2225 are configured so that they can move axially relative to one another from the initial, pre-use configuration of FIG. 17A to the operable configuration of FIG. 17B. In some embodiments, this may be accomplished by simple axial translation of the lower casing portion 2225 toward the upper casing portion 2226. In other embodiments, the casing portions 2226, 2225 may be threadably connected so that axial translation may be accomplished by rotating the lower casing 2225 relative to the upper casing 2226. In either case, the relative axial movement causes the contact members 2280 to contact and penetrate the membrane 2290 as shown in FIG. 17B. This effect may be more of a tearing action in embodiments where the translation is caused by rotation.

As shown in FIG. 17B, penetration of the membrane 2290 allows vaporizable fluid 2242 to pass into the lower reservoir chamber 2246. A fluid flow window 2232 is provided through the wall 2231 in the lower reservoir chamber 2246 to allow vaporization fluid 2242 to flow therethrough. The vaporizer 2210 has an annular, cylindrical composite wick 2250 that may be positioned so that at least a portion of its outer circumferential surface 2252 is in registration with the fluid flow window 2232. In particular embodiments, this may be a composite wick having one or more active material layers or having an active material dispersed therein. In some embodiments, the wick 2250 may be sized and positioned so that it extends into the window 2232. In some embodiments, the wick 2250 may be sized and positioned so that it extends through the window 2232 into the lower chamber 2246 of the reservoir 2240.

As in the vaporizer 2010 of FIG. 14, a coil-type heating element 2260 is disposed within the interior of the cylindrical composite wick 2250. The wick 2250 and heating element 2260 may be collectively sized and positioned so that the inner surface 2254 of the composite wick 2250 is closely adjacent or in contact with the outer surface of the heating element 2260.

Prior to first use, the vaporizer 2210 may be as shown in FIG. 17A when in the vertical orientation. In this configuration, the impermeable barrier 2290 is in position and prevents vaporizable fluid 2242 from passing into the lower chamber 2246. Accordingly, there is no communication of the fluid 2242 with the composite wick 2250. In the operational configuration of FIG. 17B, the membrane 2290 has been ruptured, allowing the vaporizable liquid 2242 to drop into the lower chamber 2246 where it passes through the window 2232 to contact the upstream surface 2252 of the wick 2250. The wick 2250 then draws the liquid 2242 to the inner surface 2254. The vaporizer 2210 may now operate in a substantially similar manner to the vaporizer 2010 of FIGS. 14 and 15.

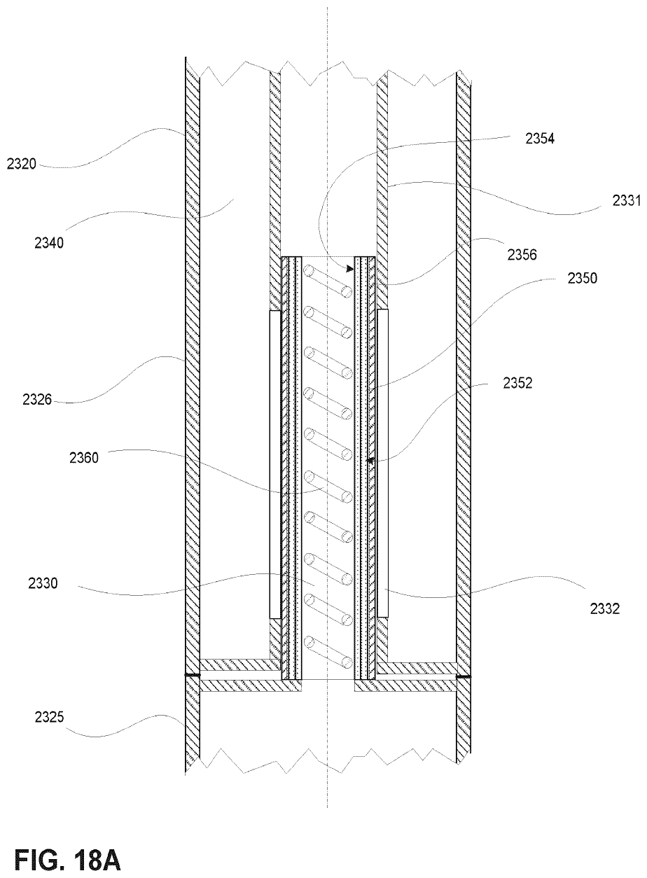

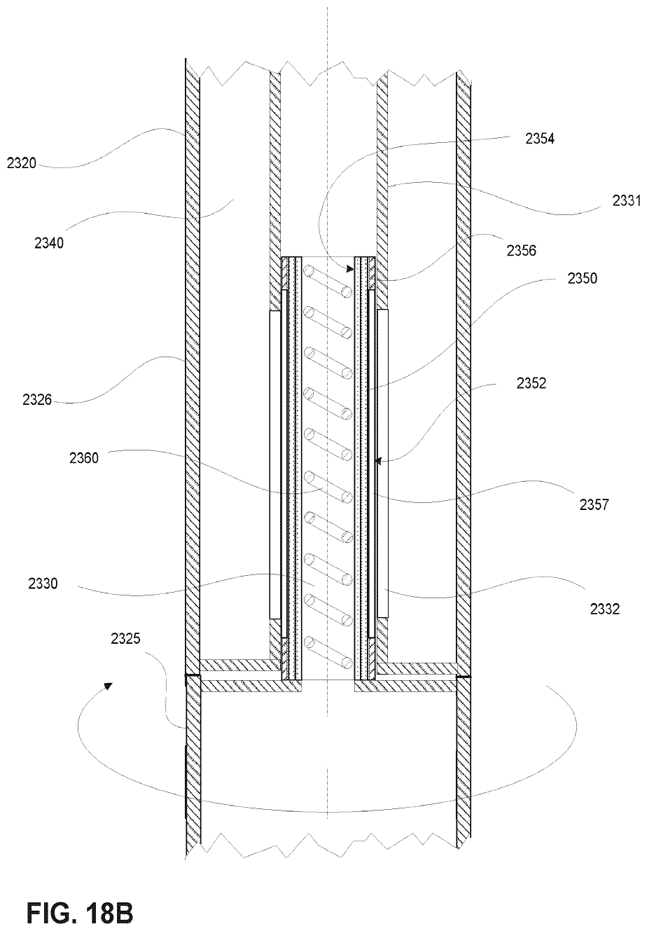

FIGS. 18A and 18B illustrate a portion of a vaporizer according to an embodiment of the invention having a cylindrical casing 2320 formed from an upper casing portion 2326 and a lower casing portion 2325. The personal vaporizer has a cylindrical fluid reservoir 2340 that surrounds the vaporization chamber 2330. One or more fluid flow windows 2332 are provided through the wall 2331 to allow vaporization fluid 2342 to flow therethrough.

The vaporizer 2310 has an annular, cylindrical composite wick 2350. In particular embodiments, this may be a composite wick having one or more active material layers or having an active material dispersed therein. The wick 2350 has a wick body that may be substantially similar to the layered cylindrical wick 700 of FIG. 9. Alternatively, the wick body may be formed as a single layer composite in which a base wick material has an active material dispersed there-within similar to what is illustrated in FIGS. 2 and 3. In this embodiment, the wick 2350 has a cylindrical outer casing 2356 that is fluid impermeable. The outer casing 2356 has casing windows 2357 around a portion of its circumference. The outer casing 2356 and the casing windows 2357 may be configured so that the outer casing 2356 may be initially positioned with the casing windows 2357 out of registration with the fluid flow windows 2332 (as in the pre-use configuration shown in FIG. 18A) and then rotated to an orientation where the casing windows 2357 are in registration with the fluid flow windows 2332 (as in the operational configuration of FIG. 18B). The outer casing 2356 is sized and configured so that when the casing windows 2357 are out of registration with the fluid flow windows 2332, the outer casing 2356 acts as a fluid flow barrier that prevents vaporizable liquid 2342 from flowing out of the reservoir 2340. When the casing windows 2357 are not in registration with the fluid flow windows 2332, the wick body is isolated from the vaporizable liquid. When the casing windows 2357 are in registration with the fluid flow windows 2332, the wick body is in fluid communication with the vaporizable liquid in the reservoir 2340.

The upper and lower casing portions 2326, 2325 may be rotatably connected to one another. The wick 2350 is attached to the lower casing portion 2325 so that when the lower casing portion 2325 is rotated relative to the upper casing portion 2326, the wick 2350 is also rotated. Thus the user can rotate the wick 2350 from the pre-use configuration shown in FIG. 18A to the operable configuration of FIG. 18B by simply rotating the lower casing portion as shown. In some embodiments, the casing portions 2325, 2326 may incorporate a locking mechanism (e.g., integrally formed detents) that prevents the casing portions 2325, 2326 from rotating back out of the operational configuration. In other embodiments, the casing portions 2325, 2326 may be remain rotatable relative to one another, thereby allowing the user to re-isolate the wick 2350 from the liquid 2342 remaining in the reservoir 2340.

As in the vaporizer 2010 of FIG. 14, a coil-type heating element 2360 is disposed within the interior of the cylindrical composite wick 2350. The wick 2350 and heating element 2360 may be collectively sized and positioned so that the inner surface 2354 of the composite wick 2350 is closely adjacent or in contact with the outer surface of the heating element 2360.

Once rotated to the operable configuration of FIG. 18B, the vaporizer may be operated in a substantially similar manner to the vaporizer 2010 of FIGS. 14 and 15.

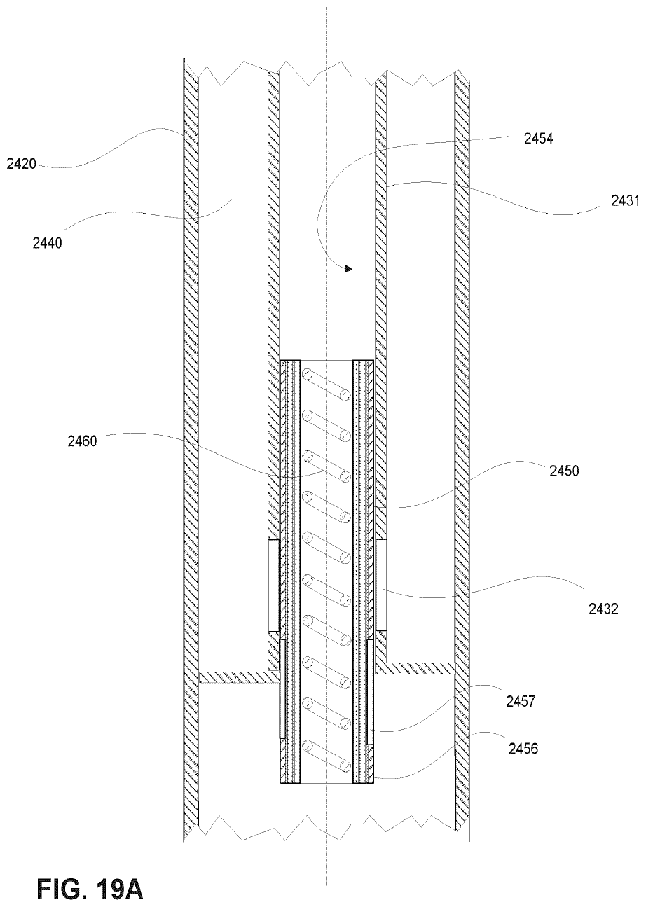

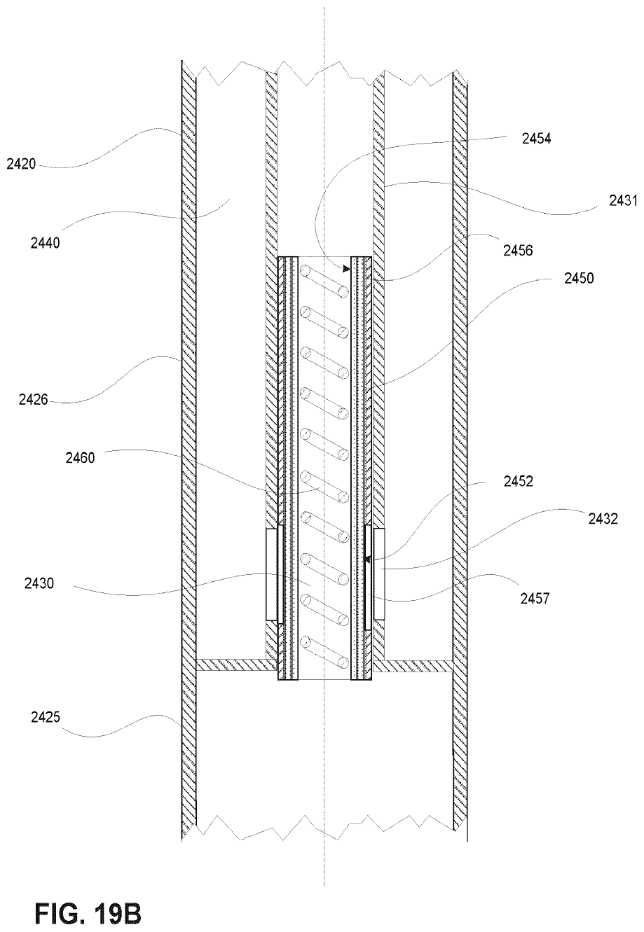

FIGS. 19A and 19B illustrate a portion of a vaporizer according to an embodiment of the invention having a cylindrical casing 2420 and a cylindrical fluid reservoir 2440 that surrounds the vaporization chamber 2430. One or more fluid flow windows 2432 are provided through the wall 2431 to allow vaporization fluid 2442 to flow therethrough.

The vaporizer 2410 has an annular, cylindrical composite wick 2450. The wick 2450 has a wick body that may be substantially similar to the layered cylindrical wick 700 of FIG. 9. Alternatively, the wick body may be formed as a single layer composite in which a base wick material has an active material dispersed there-within similar to what is illustrated in FIGS. 2 and 3. In this embodiment, the wick 2450 has a cylindrical outer casing 2456 that is fluid impermeable. The outer casing 2456 has casing windows 2457 around a portion of its circumference at a particular axial location. This wick 2450 may be configured to be translatable within the vaporization chamber from a first axial position in which the casing windows 2457 are not in registration with the fluid flow windows 2432 (as in FIG. 19A) to a second axial location where the casing windows 2457 are in registration with the fluid flow windows 2432 (as in FIG. 19B). This translation may be accomplished through any suitable mechanical means (e.g., a lever or slide attached to the wick casing 2456 and passing through a slot in the vaporizer casing 2420). The outer casing 2456 is sized and configured so that when the casing windows 2457 are out of registration with the fluid flow windows 2432, the outer casing 2456 acts as a fluid flow barrier that prevents vaporizable liquid 2442 from flowing out of the reservoir 2440. Accordingly, when the casing windows 2457 are not in registration with the fluid flow windows 2432, the wick body is isolated from the vaporizable liquid. When the casing windows 2457 are in registration with the fluid flow windows 2432, the wick body is in fluid communication with the vaporizable liquid in the reservoir 2440.

In some embodiments, a locking mechanism (e.g., integrally formed detents) may be included to prevent the fluid transport structure 2450 from being translated back to the pre-use configuration. In other embodiments, the casing portions the fluid transport structure may be remain movable, thereby allowing the user to re-isolate the wick body 2451 from the liquid 2442 remaining in the reservoir 2440.

As in the vaporizer 2010 of FIG. 14, a coil-type heating element 2460 is disposed within the interior of the cylindrical composite wick 2450. The wick 2450 and heating element 2460 may be collectively sized and positioned so that the inner surface 2454 of the composite wick 2450 is closely adjacent or in contact with the outer surface of the heating element 2460.

Once the wick 2450 has been translated to the operable configuration of FIG. 19B, the vaporizer may be operated in a substantially similar manner to the vaporizer 2010 of FIGS. 14 and 15.

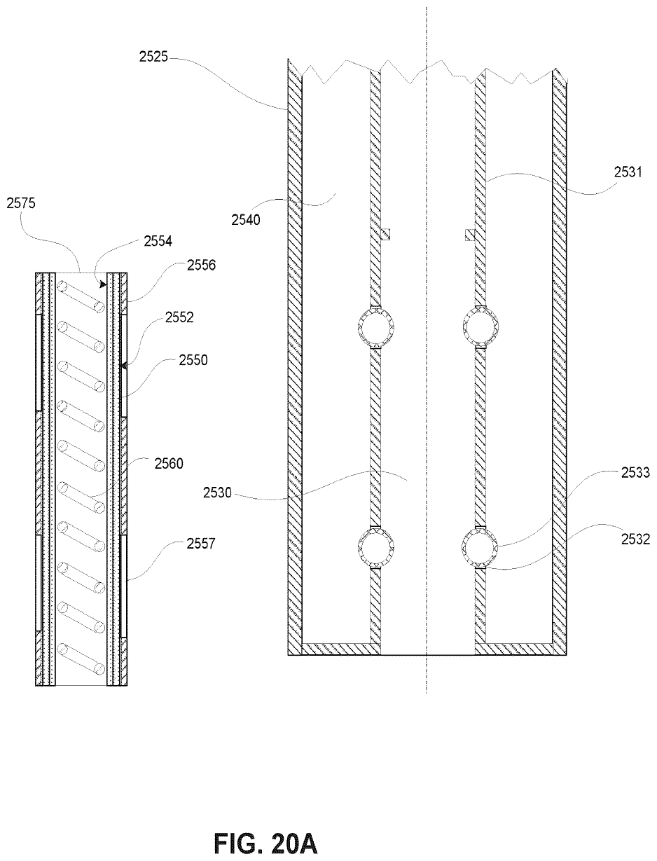

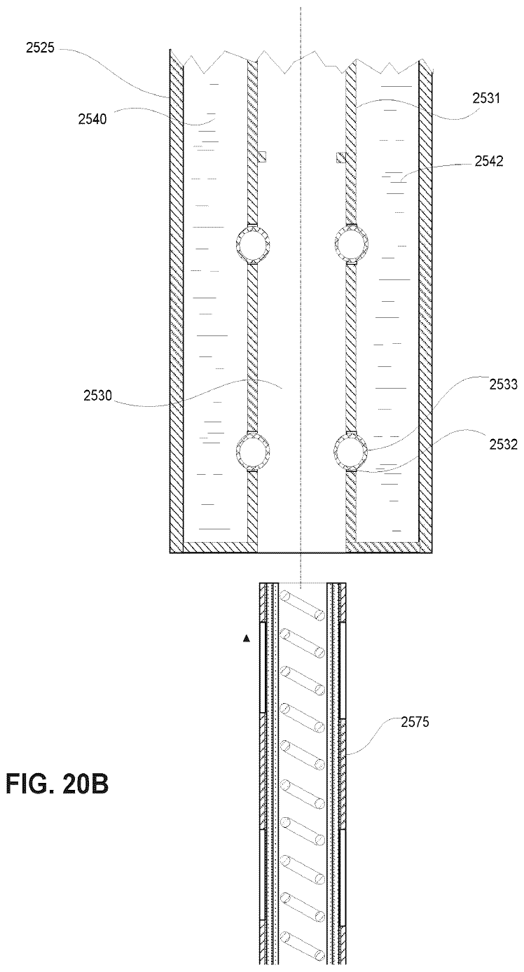

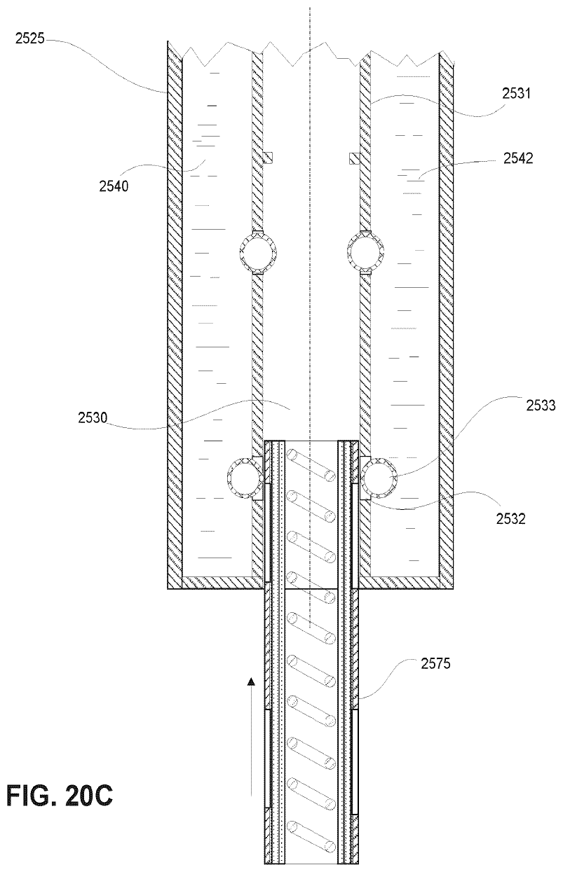

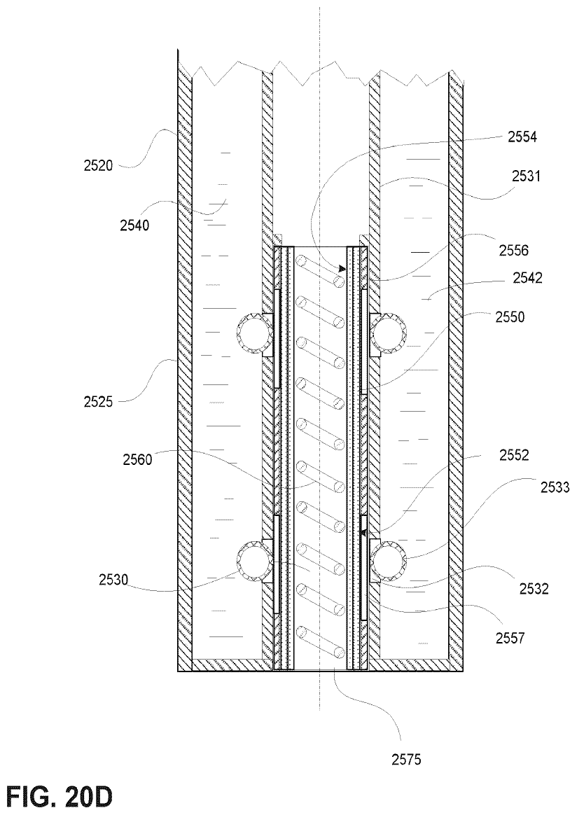

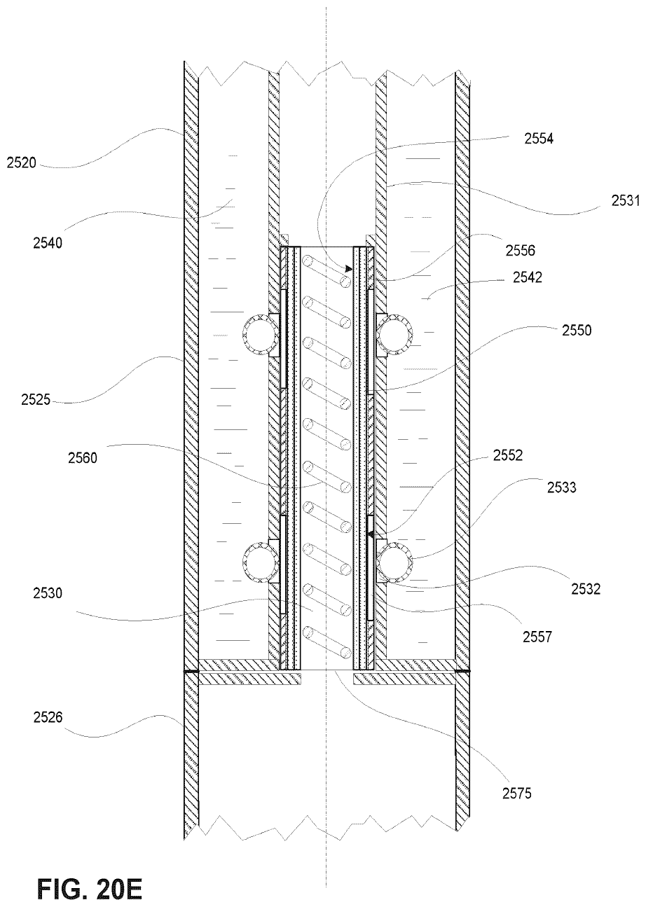

FIGS. 20A-20E illustrate a portion of a vaporizer according to an embodiment of the invention in which a composite wick 2550 is contained within a casing 2556 to provide a wick cartridge 2575 that can be kept separate from the vaporizer until use. The wick 2550 has a wick body that may be substantially similar to the layered cylindrical wick 700 of FIG. 9. Alternatively, the wick body may be formed as a single layer composite in which a base wick material has an active material dispersed there-within similar to what is illustrated in FIGS. 2 and 3. The cartridge casing 2556 is fluid impermeable, but has casing windows 2557 around a portion of its circumference at particular axial locations established to provide for alignment with fluid flow windows 2532 when the wick cartridge 2575 is installed.

The wick cartridge 2575 also includes a coil-type heating element 2560 disposed within the interior of the cylindrical composite wick 2550. The wick 2550 and heating element 2560 may be collectively sized and positioned so that the inner surface 2554 of the composite wick 2550 is closely adjacent or in contact with the outer surface of the heating element 2560. In some embodiments, the wick cartridge 2575 may include electrical contacts for connecting the heating element 2560 to a power source contained in one of the casing portions 2525, 2526 described below.

The vaporizer of this embodiment has two separate sections, an upper section with an upper casing 2525 and a lower section with a lower casing 2526. The lower section may be configured to house control and power source components, while the upper section includes the mouthpiece and exit passage. As shown in FIG. 20A, the cylindrical upper casing 2525 surrounds a cylindrical fluid reservoir 2540 that surrounds a vaporization chamber 2530. One or more fluid flow windows 2532 are provided through the vaporization chamber wall 2531 to allow vaporization fluid 2542 to flow therethrough. In this embodiment, however, the fluid flow windows are initially blocked by plug members 2533. In some embodiments, the plug members 2533 are rigid or relatively rigid and are designed to be forcibly displaced to allow fluid to pass through the windows 2532. In other embodiments, the plug members 2533 may be designed to be deformed or ruptured so as to allow fluid passage. In particular embodiments, the plug members 2533 may be toroidal rings sized to lodge within and obstruct passage through the windows 2532.

FIGS. 20B-20D illustrate a sequence in which a cartridge 2575 is installed into an upper portion of a vaporizer that has vaporizable fluid 2542 in the fluid reservoir 2540. In FIG. 20B, the cartridge 2575 is aligned with the vaporization chamber 2530. In FIG. 20C, the cartridge has been partially received into the chamber 2530. In reaching this position, the cartridge casing 2556 has engaged the lower set of plug members 2533 and forced them outward to allow fluid 2542 into the lower fluid flow windows 2532. In FIG. 20D, the cartridge is fully installed and all of the plug members 2533 have been displaced from the fluid flow windows 2532. Also, in this final position, the casing windows 2557 are in registration with the fluid flow windows 2532, thereby placing the wick 2550 in fluid communication with the vaporizable liquid in the reservoir 2540. The wick body 2551 can then transport the liquid 2542 to the inner wick body surface 2554.

Once the wick cartridge 2575 has been installed, the upper casing 2525 may be attached to the lower casing 2526 as shown in FIG. 20E. Upon doing so, the vaporizer may be operated in a substantially similar manner to the vaporizer 2010 of FIGS. 14 and 15.

In some embodiments, the plug members 2533 may be selected or configured so that the movement, displacement or deformation caused by the interaction with the cartridge 2575 is reversed upon subsequent removal of the cartridge 2575. In such embodiments, removal of the cartridge 2575 essentially returns the vaporizer 2510 to the pre-use configuration of FIG. 20A.

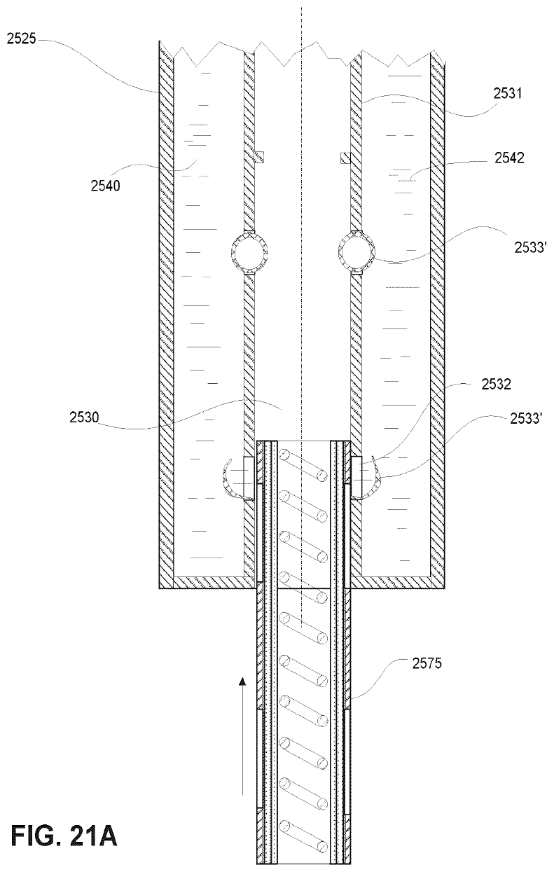

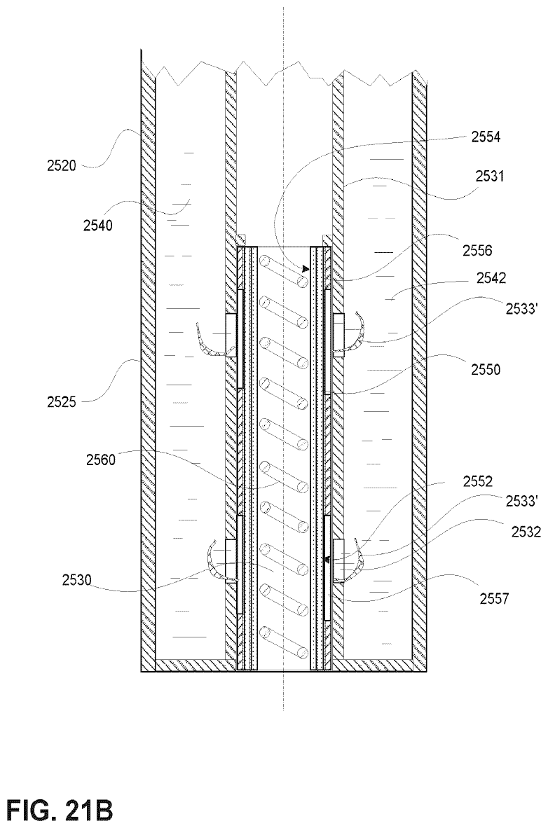

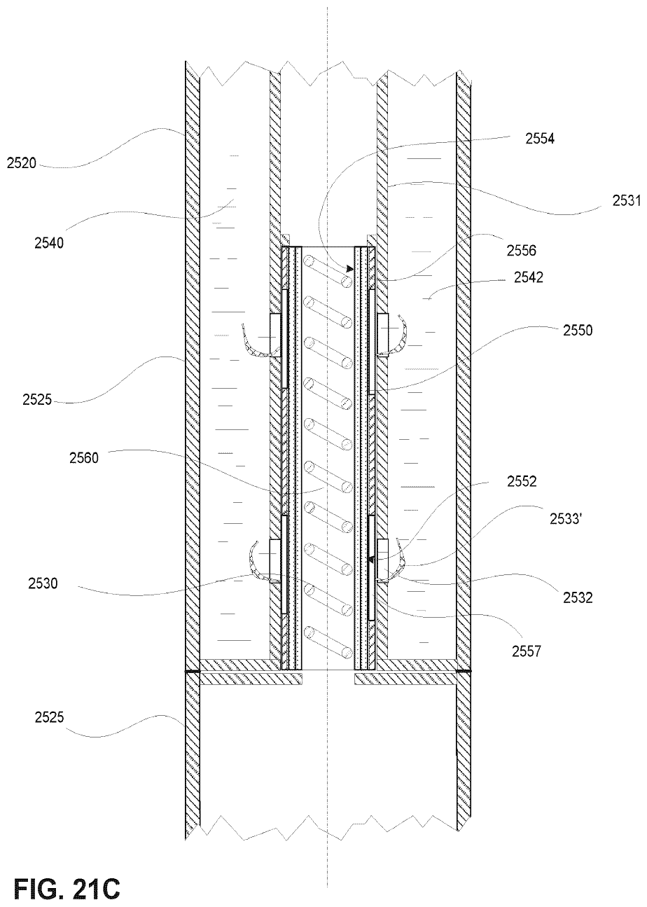

FIGS. 21A-21C illustrate a variation on the above embodiment that uses frangible plug members 2533′ in place of the displaceable plug members 2533. Installation and positioning of the cartridge 2575 are the same as previously described with the final installation configuration shown in FIG. 21B. FIGS. 20B-20E, with the final installation configuration shown in FIG. 21C. In this embodiment, the plug members 2533′ do not displace when contacted by the casing 2556 of the cartridge 2575. Instead, as shown in FIGS. 21B and 21C, when the plug members 2533′ are contacted by the casing 2556, the application of additional insertion force causes the plug members 2533′ to break, thereby allowing liquid 2542 to pass through the fluid flow windows 2532. When the cartridge 2575 is full inserted, the casing windows 2557 are in registration with the fluid flow windows 2532, thereby allowing the liquid 2542 to reach the outer surface 2552 of the wick body 2551.

As before, the upper casing 2525 may then be attached to the lower casing 2526 as shown in FIG. 21C and the vaporizer operated in a substantially similar manner to the vaporizer 2010 of FIGS. 1 and 2.

It will be understood that in variations on the above embodiments, the fluid transport cartridge 2575 could be attached to or partially disposed within the lower main body casing 2526 and that insertion into the upper body portion 2525 may be accomplished as part of the action to attach the lower casing 2526 to the upper casing 2525.

The present invention provides vaporizers having a fluid transport structure that is initially isolated from the vaporizable liquid in the fluid reservoir. Only when a user selectively changes the configuration of the vaporizer from a pre-use to an operational state is liquid allowed to contact and flow through the fluid transport structure. The invention encompasses various methods and mechanisms for isolating and de-isolating the fluid transport structure. The various embodiments of the invention all serve to mitigate or eliminate effects of prolonged exposure of transport structure component materials (e.g., wicking materials and/or active materials of composite wick structures) to vaporizable liquids.

While the foregoing illustrates and describes exemplary embodiments of this invention, it is to be understood that the invention is not limited to the construction disclosed herein. The invention can be embodied in other specific forms without departing from the spirit or essential attributes.

1. A personal vaporizer comprising:
a main body having a main body wall defining a main body interior;
a vaporization chamber within the main body interior, the vaporization chamber being defined at least in part by a vaporization chamber wall having a fluid flow window formed therethrough;
an exit chamber extending from the vaporization chamber to an exit passage in communication with an atmosphere exterior to the main body;
a heating element disposed within the vaporization chamber;
a fluid reservoir having a reservoir interior defined at least in part by the vaporization chamber wall, the fluid reservoir being configured for storage of a vaporizable liquid therein and for passage of the vaporizable liquid through the fluid flow window formed through the vaporization chamber wall when the personal vaporizer is in an operational configuration;
a fluid transport structure comprising a wick body at least partially disposed within the vaporization chamber and configured for drawing vaporizable liquid through an upstream wick surface adjacent or disposed within the fluid flow window when the personal vaporizer is in the operational configuration and for transporting the vaporizable liquid to a downstream wick surface in contact with or adjacent the heating element; and
one or more impermeable fluid barriers configured and positioned to prevent vaporizable liquid from passing through the fluid flow window when the personal vaporizer is in a pre-use configuration and for allowing vaporizable liquid to pass through the fluid flow window and contact the upstream wick surface when the personal vaporizer is in the operational configuration.

2. A personal vaporizer according to claim 1, wherein the personal vaporizer can be selectively changed from the pre-use configuration to the operational configuration by at least one of the set consisting of removal, displacement, distortion, or breach of at least one of the one or more impermeable fluid barriers.

3. A personal vaporizer according to claim 1, wherein:
the main body is cylindrical, has a main body longitudinal axis, and is positionable in a vertical orientation in which the main body longitudinal axis is vertically aligned,
the vaporization chamber is cylindrical and has a longitudinal chamber axis that is coaxial with the main body longitudinal axis,
the wick body is an annular cylinder surrounding the heating element with the downstream surface being an inner cylindrical surface of the annular cylinder that is in contact with or adjacent the heating element and the upstream surface is an outer cylindrical surface of the annular cylinder,
the fluid reservoir has an upper reservoir portion and a lower reservoir portion, the lower reservoir portion being in fluid communication with the fluid flow window through the cylindrical vaporization chamber wall, and
the one or more impermeable fluid barriers comprises a fluid impermeable barrier member positioned between the upper and lower reservoir portions and configured to prevent vaporizable liquid in the upper reservoir portion from flowing into the lower reservoir portion until the fluid impermeable barrier member is removed or breached.

4. A personal vaporizer according to claim 3, wherein a tab portion of the fluid impermeable barrier member extends through an opening in the case wall and the fluid impermeable barrier member is configured so that it can be removed from the main body by grasping the tab portion and pulling the barrier member through the opening.

5. A personal vaporizer according to claim 3, wherein the fluid impermeable barrier member is a flexible membrane.

6. A personal vaporizer according to claim 5, further comprising penetration means configured for selectively breaching the fluid impermeable barrier member.

7. A personal vaporizer according to claim 3, wherein:
the main body has an upper body portion in which the upper reservoir portion is disposed and a lower body portion in which the lower reservoir portion is disposed, the lower body portion being axially movable relative to the upper body portion from a pre-use position to a use position,
the fluid impermeable barrier member is fixedly attached to the upper body portion, and
the personal vaporizer further comprises:
at least one barrier contact member attached to the lower body portion, the at least one barrier contact member being configured and positioned so that it is spaced apart from the fluid impermeable barrier member when the lower body portion is in the pre-use position and so that when the lower body portion is moved to the use position, the at least one barrier contact member contacts and breaches the fluid impermeable barrier member, thereby permitting vaporizable liquid to flow from the upper reservoir portion into the lower reservoir portion.

8. A personal vaporizer according to claim 7, wherein the lower body portion is threadably attached to the upper body portion so that axial motion of the lower body portion relative to the upper body portion is accomplished by rotation of the lower body portion relative to the upper body portion.

9. A personal vaporizer according to claim 7, wherein the lower body portion is at least partially telescopically receivable into the upper body portion.

10. A personal vaporizer according to claim 1, wherein:
the main body is cylindrical, has a main body longitudinal axis, and has an upper body portion having an upper body wall defining an upper body interior and a lower body portion having a lower body wall defining a lower body interior, the lower body portion being movably attached to the upper body portion to allow movement of the lower body portion relative to the upper body portion from a pre-use position to a use position,
the vaporization chamber is cylindrical and has a longitudinal chamber axis that is coaxial with the main body longitudinal axis,
the wick body is an annular cylinder surrounding the heating element with the downstream surface being an inner cylindrical surface of the annular cylinder that is in contact with or adjacent the heating element and the upstream surface is an outer cylindrical surface of the annular cylinder,
the fluid transport structure further comprises a cylindrical wick casing surrounding the wick body and having at least one wick casing window formed therethrough, the wick casing being attached to the lower body portion and configured so that when the lower case portion is in the pre-use position, the at least one wick casing window is out of registration with the at least one fluid flow window and so that when the lower body portion is moved to the use position, the at least one wick casing window moves into registration with the at least one fluid flow window.

11. A personal vaporizer according to claim 10, further comprising:
a locking mechanism configured to engage the lower body portion when the lower body portion is moved into the use position and to prevent movement of the lower body portion away from the use position.

12. A personal vaporizer according to claim 10, wherein the lower body portion is movable from the pre-use position to the use position by rotating the upper body portion relative to the lower body portion.

13. A personal vaporizer according to claim 10, wherein the lower body portion is movable from the pre-use position to the use position by translating the upper body portion relative to the lower body portion along the longitudinal axis.

14. A personal vaporizer comprising:
a wick cartridge comprising:
a cylindrical wick casing having at least one wick casing window formed therethrough;
an annular wick body disposed within the cylindrical wick casing, the annular wick body having an inner cylindrical surface and an outer cylindrical surface and being configured for receiving vaporizable fluid through the outer cylindrical surface and conducting it through the cylindrical wick body to the inner cylindrical surface; and
a heating element at least partially disposed within the annular wick body so that at least a portion of the heating element is in contact with or adjacent the inner cylindrical surface of the annular wick body;
a main vaporizer body having a main body wall defining a main body interior and having a longitudinal axis;
a vaporization chamber within the main body interior, the vaporization chamber being defined by a cylindrical vaporization chamber wall surrounding the longitudinal axis and having at least one fluid flow window formed therethrough, the vaporization chamber being configured for receiving the wick cartridge so that the at least one fluid window is in registration with the at least one wick casing window when the wick cartridge is fully received into an installed position within the vaporization chamber;
a fluid reservoir surrounding at least a portion of the vaporization chamber and configured for storage of a vaporizable liquid therein, the fluid reservoir being in fluid communication with the at least one fluid flow window; and
a plug member for each of the at least one fluid flow window, each plug member being positioned at least partially within its respective fluid flow window and extending into the vaporization chamber and being configured to prevent fluid flow through its respective fluid flow window until the wick cartridge has been received into the vaporization chamber.

15. A personal vaporizer according to claim 14, further comprising:
a locking mechanism configured to engage and retain the wick cartridge when the wick cartridge is in the installed position.

16. A personal vaporizer according to claim 14, wherein each plug member is configured and positioned so that when the wick cartridge is received into the vaporization chamber, the wick casing contacts the plug member and at least partially displaces the plug member such that fluid is allowed to pass through the at least one fluid flow window.

17. A personal vaporizer according to claim 14, wherein each plug member is configured and positioned so that when the wick cartridge is received into the vaporization chamber, the wick casing contacts the plug member and at least partially distorts the plug member such that fluid is allowed to pass through the at least one fluid flow window.

18. A personal vaporizer according to claim 14, wherein each plug member comprises a frangible portion configured and positioned so that when the wick cartridge is received into the vaporization chamber, the wick casing contacts and fractures the frangible portion so that fluid is allowed to pass through the at least one fluid flow window.

19.-36. (canceled)
